(19) United States
(12) Patent Application Publication
(10) Pub. No.: US 2006/0194814 A1
(43) Pub. Date: Aug. 31, 2006

(54) Benzamide compounds

(76) Inventors: Vassilios Papadopoulos, North Potomac, MD (US); Janet Greeson, Las Vegas, NV (US); Laurent Lecanu, McLean, VA (US)

Correspondence Address: Schwegman, Lundberg, Woessner & Kluth, P.A., P.O. Box 2938, Minneapolis, MN, 55402, US

(21) Appl. No.: 11/292,797

(22) Filed: Dec. 2, 2005

Related U.S. Application Data
(63) Continuation of application No. PCT/US04/15791, filed on May 20, 2004.
(63) Continuation of application No. PCT/US05/14131, filed on Apr. 22, 2005.
(63) Continuation of application No. PCT/US04/16126, filed on May 20, 2004.
(60) Provisional application No. 60/474,964, filed on Jun. 2, 2003.
(60) Provisional application No. 60/475,642, filed on Jun. 4, 2003.
(60) Provisional application No. 60/478,648, filed on Aug. 1, 2003.
(60) Provisional application No. 60/564,636, filed on Apr. 22, 2004.
(60) Provisional application No. 60/564,636, filed on Apr. 22, 2004.
(60) Provisional application No. 60/592,688, filed on Jul. 30, 2004.

(51) Int. Cl. A61K 31/496

(52) U.S. Cl. 514/254.09

(57) ABSTRACT

The invention provides a therapeutic method for preventing or treating a pathological condition or symptom in a mammal, such as a human, wherein the infectivity of a pathogen such as a retrovirus toward mammalian cells is implicated and inhibition of its infectivity is desired comprising administering to a mammal in need of such therapy, an effective amount of an N-benzamide derivative of a piperazinyl amide of an amino acid thereof that inhibits pathogenic infectivity, including pharmaceutically acceptable salts thereof. The invention also provides a therapeutic method for preventing or treating a neuropathological condition or symptom in a mammal, such as human, comprising administering to a mammal in need of such therapy, an effective amount of an N-benzamide derivative of a piperazinyl amide of an amino acid thereof, including pharmaceutically acceptable salts thereof.

### RELATED APPLICATIONS

This application is a continuation under 35 U.S.C. 111(a) of:

1) International Application No. PCT/US2004/015791 filed May 20, 2004 and published in English as WO 2004/108076 A2 on Dec. 16, 2004, which claims the benefit of U.S. Provisional Application Nos. 60/474,964 filed Jun. 2, 2003; 60/475,642 filed Jun. 4, 2003; 60/478,648 filed Aug. 1, 2003; and 60/564,636 filed Apr. 22, 2004; and

2) International Application No. PCT/US2005/014131 filed Apr. 22, 2005, which claims the benefit of U.S. Provisional Application No. 60/564,636 filed Apr. 22, 2004; International Application No. PCT/US2004/015791 filed May 20, 2004 and published in English as WO 2004/108076 A2 on Dec. 16, 2004; International Application No. PCT/US2004/016126 filed May 20, 2004 and published in English as WO 2005/000205 on Jan. 6, 2005; and 60/592,688 filed Jul. 30, 2004;

which applications and publications are incorporated herein by reference.

### BACKGROUND OF THE INVENTION

The global HIV/AIDS epidemic killed more than 3 million people in 2003, and an estimated 5 million acquired the human immunodeficiency virus (HIV)—bringing to 40 million the number of people living with the virus around the world. Despite progress in developing anti-viral regimens, there is not a fully effective therapy for AIDS. Current therapeutic strategies for AIDS include protease inhibitors, nucleoside analog reverse transcriptase inhibitors, non-nucleoside analog reverse transcriptase inhibitors, fusion inhibitors and also the highly toxic hydroxyurea (Yarchoan R et al. (1986) Lancet 1(8481): 575-580; Richards A D et al. (1989) FEBS Lett 247(1): 113-117; Gao W Y et al. (1995) Proc Natl Acad Sci USA 92(18): 8333-8337; De Clercq E (1999) Farmaco 54(1-2): 26-45; Williams I G (2003) Int J Clin Pract 57(10): 890-897). Unfortunately, emerging resistances due to virus genotype mutations (Cavert W and Balfour HH (2003) Clin Lab Med 23(4): 915-928; Gallant J E et al. (2003) Antivir Ther 8(6): 489-506; Olson W C and Maddon P J (2003) Curr Drug Targets Infect Disord 3(4): 283-294) and serious side-effects are strong limitations to the treatment efficacy.

Currently, there is a need for effective anti-viral agents, including anti-retroviral agents. There is also a need for pharmacological tools for the further study of physiological processes associated with infection.

Alzheimer's disease (AD) is the most common dementia occurring in elderly, affecting about 10% of people above 65 years and 40% above 80 years. The familial AD is the early-onset form of the disease that involves different mutations of the amyloid protein precursor (APP) gene and accounts for no more than 5% of the total AD cases. The late-onset form of the disease, also called sporadic form, accounts for more than 95% of the AD cases and its origins remain elusive. Several risk factors have been identified or are suspected. These include the ε4 allele of the apoE gene, socio-economical situation or previous medical conditions, but a causality relationship of the onset or progression of the disease has not been yet established.

AD is clinically characterized by a progressive and irreversible impairment of cognition processes and memory alteration, and is commonly associated with a non-cognitive symptomatology, including depression (Robert et al., Alzheimer's Disease: from molecular biology to therapy, R. Becker et al., eds., (1996) at 487-493. Alzheimer's disease (AD) neuropathology is histologically characterized by an increase of brain β-amyloid (AP) peptide levels accompanied by the formation of senile plaques (Nikaido et al. (1970) Trans Am. Neurol. Assoc. 95:47-50 and the appearance of neurofibrillary tangles (NFT), due to a hyperphosphorylation of the Tau protein (Kosik et al., (1986) PNAS USA 83:4044-8. Aβ is produced by proteolytic cleavage of the 3-amyloid precursor protein (β-APP) by the membrane enzymes β- and γ-secretase. Aβ exists either as the most commonly found 40 amino acid length Aβ₁₋₄₀ form on the 42 amino acid Aβ₁₋₄₂ form, reported to be more neurotoxic than Aβ₁₋₄₀. Although understanding of Aβ-medicated neurotoxicity has dramatically increased during the last decade, no Aβ₁₋₄₂ targeting therapeutic strategy has been shown to successfully slow down the progression of the disease. Rather, current therapeutic strategies under investigation for AD include inhibitors of Aβ production, compounds that prevent its oligomerization and fibrillization, anti-inflammatory drugs, inhibitors of cholesterol synthesis, antioxidants, neurorestorative factors and vaccines (Selkoe, D. J. (1999) Nature 399, A23-31; Emilien, G., et al. (2000) Arch. Neurol. 57, 454-459; Klein, W. L. (2002) Neurochem. Internat. 41, 345-52; Helmuth, L. (2002) Science 297(5585), 1260-21.)

HIV-associated dementia (also known as HIV-Associated Dementia Complex, HIV-associated cognitive/motor complex, and AIDS Dementia Complex) is a progressive neurological disorder that affects approximately 58,000 individuals infected with the Human Immunodeficiency Virus (HIV) in the United States. HIV-associated dementia is thought to be a subcortical dementia characterized by cognitive, motor and behavioral impairments severe enough to interfere with an individual's ability to function occupationally or socially. Early manifestations of HIV-associated dementia may be characterized by cognitive impairment, loss of motor skills, and/or behavioral challenges:

Cognitive Impairment: Memory loss, impaired concentration, and mental slowing characterized by such actions as slow response are common attributes associated with cognitive impairment.

Loss of Motor Skills: Individuals experiencing difficulty with their balance, lack of coordination, leg weakness, clumsiness, poor gait, and/or deteriorating handwriting may be showing signs of deteriorating motor skills.

Behavioral Challenges: Uncharacteristic behavior, poor decision-making, personality and mood changes, and possibly psychotic behavior characterize the behavioral challenges experienced by some individuals.

Individuals suffering from HIV-associated dementia may develop these characteristics at various times and rates during the progression of the disease. HIV-associated dementia patients typically experience a high incidence of premature mortality due to or associated with their dementia. Dementia is a debilitating disease that literally steals the livelihood of its victims. Memory loss, depression, agitation, anxiety, and other adverse behaviors are caused by its apparently irreversible and destructive effects on the central nervous system. These debilitating effects further reduce the life expectancy of HIV infected individuals. Working in concert, and without effective treatment, the virus and the dementia condition, destroy individual's immune systems, self-confidence, motor skills, and family relations. As a result, individuals with HIV-associated dementia experience premature mortality.

In the absence of dementia, treatments for HIV affected individuals are given an opportunity to be more effective and possibly prolong the life of the individual. Further, in the absence of this condition the treatment may prove effective in retarding the replication of HIV and retarding its adverse effects.

HIV, the virus whose progression leads to Acquired Immune Deficiency Syndrome (AIDS), is a retrovirus housed in a viral particle protected by various coat proteins, the most significant of which is glycoprotein 120 (gp120). The gp120 envelope facilitates infection of a host cell by binding to receptors on the surface of many immune cells such as T-cells as well as chemokine co receptors. After fusion of the viral particle with the host cell, replication of the viral particles is initiated and subsequent infection of other cells occurs.

In addition to facilitating the introduction of HIV into host cells, research demonstrates that gp120 is either directly or indirectly responsible for initiating HIV dementia. The direct hypothesis suggests that the gp120 protein, which is often shed from the HIV virus after fusion occurs, interacts directly with chemokine receptors on the surface of neurons; thereby facilitating apoptosis and neuronal cell death. (Brew, Bruce James 1999). The indirect hypothesis suggests that apoptosis is caused by interaction of the HIV virus with non-neuronal cells of the central nervous system (CNS), specifically macrophages, microglia, and astrocytes. In this case, gp120 facilitates the transport of HIV infected macrophages and microglia across the blood brain barrier (BBB), a selectively permeable membrane that prevents entry of foreign material. (Kaul, Marcus, et al. 2001). Once infected cells are in the brain, they release neurotoxins and promote a massive influx of calcium ion (Ca²⁺) into the neuron thus initiating apoptosis. (Smits, H. A. et al. 2000). HIV Infected macrophages, monocytes and microglia all release gp120. An abundance of gp120 in the CNS disrupts the calcium homeostasis (Lipton S A, 1994) partly by reverting the glutamate uptake systems and by directly activating the NMDA subtype calcium channel-associated glutamatergic receptor and the calcium voltage-operated channels (Lipton S A, 1991). The induced massive calcium inward current leads to an impairment of the memory and learning processes and triggers the excitotoxicity cascade which leads to a neuronal death (Choi D W, 1992). Calcium ions facilitate intercellular communication through electrical polarization and depolarization and therefore opening a Ca²⁺ channel for too long is fatal to a neuron. (Epstein L and Gendelman H. May 1993).

A combination of both the direct and indirect interference of gp120 with the calcium homeostasis may cause mitochondrial function impairment leading to critical cell death. (Simpson, David M.). At the same time, gp120 indirectly induces an increase in blood and CSF cortisol concentrations leading to neurotoxicity and HIV-associated dementia. (Corley P A. 1995; Corley P A. 1996).

Chemokine receptors are also bound by the gp120 envelope as co receptors with CD4 to permit entry into host cells. (Miller, Richard J. and Meucci, Olimpia 1999). This binding on cells of the CNS acts to stimulate and agonize the cells in an uncontrolled manner. Over stimulation subsequently acts to release glutamate and other neurotoxins and inflammatory cytokines resulting in neuronal death due to apoptosis. (Miller, Richard J. and Meucci, Olimpia. 1999).

Astrocytosis, proliferation of astrocytes, observed in patients with HIV, occurs when the virus retards the effectiveness of astrocytes to scavenge excess glutamate produced by infected macrophages and microglia. (Kaul, Marcus et al. 2001). Additional astrocytes are produced to compensate for the ineffectiveness of the cells. As a result of astrocytosis, more infected macrophages and microglia cross the BBB inducing massive neuronal death which leads to HIV-associated dementia.

It is clear that the cause of HIV-associated dementia revolves around the activation of macrophages, microglia, chemokine receptors, and astrocytes within the CNS and subsequent apoptosis leading to dementia. It is equally apparent that the process is made possible because the gp120 envelope facilitates transfer of the HIV virus across the BBB and because cleaved gp120 protein is able to interact with chemokine receptors on the surface of neurons.

These is a need for additional treatments of neuropathological disorders, including Alzheimer's, vascular dementia and/or HIV-associated dementia.

### SUMMARY OF THE INVENTION

The invention provides a method to prevent viral replication by blocking or inhibiting the ability of viruses, such as retroviruses, including HIV, to infect mammalian cells in vitro or in vivo. Thus, the present invention provides a method for treatment of a mammal threatened or afflicted by an infectious pathogen, by administering to said mammal an effective amount of a compound of formula I: 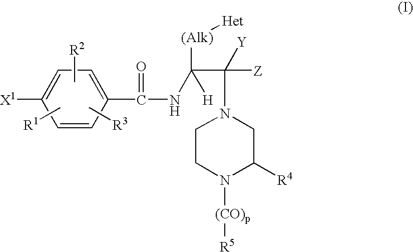
wherein:

a) R¹, R², R³, R⁴ and R⁵ are individually H, OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio or (C₁-C₆)alkanoyloxy; or R¹ and R² together are methylenedioxy;

b) X¹ is NO₂, CN, —N═O, (C₁-C₆)alkylC(O)NH—, isoxazolyl, or N(R⁶)(R⁷) wherein R⁶ and R⁷ are individually, H, (C₁-C₆)alkyl, (C₂-C₆)alkenyl, (C₃-C₆)cycloalkyl, (C₃-C₆)cycloalkyl((C₁-C₆)alkyl), wherein cycloalkyl optionally comprises 1-2, S, nonperoxide O or N(R⁸), wherein R⁸ is H, (C₁-C₆)alkyl, (C₃-C₆)cycloalkyl, (C₃-C₆)cycloalkyl(C₁-C₆)alkyl or benzyl; aryl, aryl(C₁-C₆)alkyl, aryl(C₂-C₆)alkenyl, heteroaryl, heteroaryl(C₁-C₆)alkyl, or R⁶ and R⁷ together with the N to which they are attached form a 5- or 6-membered heterocyclic or heteroaryl ring, optionally substituted with R¹ and optionally comprising 1-2, S, non-peroxide O or N(R⁵);

c) Alk is (C₁-C₆)alkyl;

d) Y and Z together are ═O, —O(CH₂)ₘO— or —(CH₂)ₘ— wherein m is 2-4, or Y is H and Z is OH or SH;

e) Het is heteroaryl or heterocycloalkyl, each optionally substituted by 1, 2 or 3 of R¹ or a combination thereof or is a bond connecting (Alk) to NH;

f) p is 0 or 1; or the pharmaceutically acceptable salt thereof.

The invention also provides a method to treat a neuropathlogical condition including a central nervous system (including, for example, stroke, brain, retina and/or spinal cord injuries, ischemia and reperfusion, and other brain or retinal disorders, and trauma associated with neurosurgical procedures) disease or disorder; cognitive impairment; a psychiatric disorder, including depression and mood alteration; acquired immunodeficiency syndrome; multiple sclerosis; HIV-associated dementia, vascular dementia, Alzheimer's disease; Huntington's disease; epilepsy; lathyrism; amyotrophic lateral sclerosis; Parkinson's disease; and cancer, including, for example brain cancer. Thus, the present invention provides a method for treatment of a mammal threatened or afflicted by a neuropathological condition by administering to said mammal an effective neuroprotective amount of a compound of formula I: 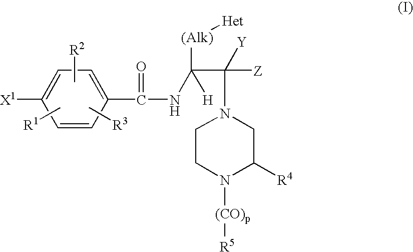
wherein:

a) R¹, R², R³, R⁴ and R⁵ are individually H, OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio or (C₁-C₆)alkanoyloxy; or R¹ and R² together are methylenedioxy;

b) X¹ is, NO₂, CN, —N═O, (C₁-C₆)alkyl(C(O)NH—, isoxazolyl, or N(R⁶)(R⁷) wherein R⁶ and R⁷ are individually, H, (C₁-C₆)alkyl, (C₂-C₆)alkenyl, (C₃-C₆)cycloalkyl, (C₃-C₆)cycloalkyl(C₁-C₆)alkyl), wherein cycloalkyl optionally comprises 1-2, S, nonperoxide O or N(R⁸), wherein R⁸ is H, (C₁-C₆)alkyl, (C₃-C₆)cycloalkyl, (C₃-C₆)cycloalkyl(C₁-C₆)alkyl or benzyl; aryl, aryl(C₁-C₆)alkyl, aryl(C₂-C₆)alkenyl, heteroaryl, heteroaryl(C₁-C₆)alkyl, or R⁶ and R⁷ together with the N to which they are attached form a 5- or 6-membered heterocyclic or heteroaryl ring, optionally substituted with R¹ and optionally comprising 1-2, S, non-peroxide O or N(R⁵);

c) Alk is (C₁-C₆)alkyl;

d) Y and Z are ═O, —O(CH₂)ₘO— or —(CH₂)ₘ— wherein m is 2-4, or Y is H and Z is OH or SH;

e) Het is heteroaryl or heterocycloalkyl, each optionally substituted by 1, 2 or 3 of R¹ or a combination thereof or is a bond connecting (Alk) to NH;

f) p is 0 or 1; and the pharmaceutically acceptable salts thereof.

### BRIEF DESCRIPTION OF THE FIGURES

FIG. 1 depicts the chemical structure of SP01, SP010 and SP100.

FIG. 2, panels A-C are graphs depicting the inhibitory effect of SP01, SP010 and SP100 on the HIV-1 IIIB strain replication in HeLa cells. Compounds were tested either alone or in a formulation (1A, 010A or 100A) 3TC, ddl and AZT are known anti-viral compounds.

FIG. 3, (panels A-C) are graphs depicting the inhibitory effect of 24-hour SP01, SP010 and SP100 premedication on the HIV-1 IIIB strain replication in HeLa cells. Compounds were tested in a formulation (01A, 010A or 100A).

FIG. 4 (panels A-C) are graphs depicting the inhibitory effect of 48-hour SP01, SP010 and SP100 premedication on the HIV-1 IIIB strain replication in HeLa cells.

FIG. 5 (panels A-C) are graphs depicting the inhibitory effect of SP01, SP01A and SP010 on the multi-drug resistant HIV MDR-769 strain replication in HeLa cells.

FIG. 6 is a reaction scheme for the synthesis of SP010.

FIG. 7 depicts the chemical formula of several benzoic acid derivatives including procaine and several procaine derivatives of the present invention.

FIG. 8A is a bar graph depicting the effect of procaine and SP-10 on the dbc-AMP-induced 20á-hydroxyprogesterone synthesis in pg/well compared to control in Y1 mouse adrenal tumor cells.

FIG. 8B is a bar graph depicting the effect of procaine and SP-10 on cell viability compared to control in dbc-AMP induced Y1 mouse adrenal tumor cells.

FIG. 8C is a bar graph depicting the effect of SP014, SP016, and SP017 on the dbc-AMP-induced 20á-hydroxyprogesterone synthesis in inhibition percentage in Y1 mouse adrenal tumor cells.

FIG. 8D is a bar graph depicting the effect of SP014, SP016, and SP-17 on cell viability compared to control in dbc-AMP-induced Y1 mouse adrenal tumor cells.

FIG. 9A is a bar graph depicting the effect of procaine on the dbc-AMP-induced cortisol synthesis in H295R human adrenal tumor cells.

FIG. 9B is a bar graph depicting the effect of procaine on cell viability in dbc-AMP-induced H295R human adrenal tumor cells.

FIG. 10A is a bar graph depicting the effect of procaine on the dbc-AMP-induced progesterone synthesis in MA-10 mouse Leydig tumor cells.

FIG. 10B is a bar graph depicting the effect of procaine on cell viability in dbc-AMP-induced MA-10 mouse Leydig tumor cells.

FIG. 11 is graph depicting the effect of a procaine-based formulation on serum corticosterone levels in male Sprague-Dawley rats.

FIG. 12 is a graph depicting the effect of procaine on the dbc-AMP-induced increase of the PKA activity.

FIG. 13A is a bar graph depicting the effect of procaine on hydroxycholesterol induced 20á-hydroxyprogesterone synthesis.

FIG. 13B is an immunoblot depicting the effect of procaine on the dbc-AMP-induced expression of the P450_{scc} enzyme.

FIG. 13C is an immunoblot depicting the effect of procaine on the dbc-AMP-induced StAR expression.

FIG. 14A is a bar graph depicting the effect of procaine on dbcAMP and mevalonate supported 20á-hydroxyprogesterone formation in Y1 cells.

FIG. 14B is a bar graph depicting the effect of procaine on HMG-CoA reductase activity in Y1 cells treated with dbcAMP (** p<0.01 *** p<0.001, mean±SD). 100% activity corresponds to 163±16 pmol/min/mg protein.

FIG. 15A is bar graph depicting the effect of procaine on HMG-CoA reductase mRNA expression levels by Q-PCR in dbcAMP induced versus control Y1 cells.

FIG. 15B is bar graph depicting the effect of procaine on HMG-CoA reductase mRNA expression levels by Q-PCR in dbcAMP induced versus control UT-1 cells.

FIG. 15C is bar graph depicting the effect of procaine on HMG-CoA reductase mRNA expression levels by Q-PCR in dbcAMP induced versus control Hepa1-6 mouse liver hepatoma cells.

### DETAILED DESCRIPTION

Definitions

Unless otherwise stated, the following terms used in the specification and claims are defined for the purposes of this application and have the meanings given below:

The use of the term “about” in the present disclosure means “approximately,” and illustratively, the use of the term “about” indicates that dosages outside the cited ranges may also be effective and safe, and such dosages are also encompassed by the scope of the present claims.

“Bioavailability” refers to the extent to which an active moiety (drug or metabolite) is absorbed into the general circulation and becomes available at the site of drug action in the body.

The term “derivative” refers to a compound that is produced from another compound of similar structure by the replacement of substitution of one atom, molecule or group by another. For example, a hydrogen atom of a compound may be substituted by alkyl, acyl, amino, etc., or an oxygen atom may be substituted by a nitrogen to produce a derivative of that compound.

“Drug absorption” or “absorption” refers to the process of movement from the site of administration of a drug toward the systemic circulation, for example, into the bloodstream of a subject.

An “effective amount” or “therapeutically effective amount” refers to the amount of the compound which is required to confer therapeutic effect on the treated subject.

The term “measurable serum concentration” means the serum concentration (typically measured in mmol, imol, nmol, mg, mg, or ng of therapeutic agent per ml, dl, or l of blood serum) of a therapeutic agent absorbed into the bloodstream after administration.

“Metabolism” refers to the process of chemical biotransformations of drugs in the body.

The term “pharmaceutically acceptable” is used adjectivally herein to mean that the modified noun is appropriate for use in a pharmaceutical product.

As used herein, the terms “pharmaceutically acceptable carrier” or “pharmaceutically acceptable excipients” includes any and all solvents, dispersion media, coatings, antibacterial and antifungal agents, preservative and antioxidative agents, isotonic and absorption delaying agents, and the like. The use of such media and agents for ingestible substances is well known in the art. Except insofar as any conventional media or agent is incompatible with the compositions, its use is contemplated.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasma concentration” refers to the concentration of a substance in blood plasma or blood serum.

“Plasma half-life” refers to the time required for the plasma drug concentration to decrease by 50% from its maximum concentration.

The term “prevent” or “prevention,” in relation to a cortisol-mediated disease or disorder in a subject, means no disease or disorder development if none had occurred, or no further disorder or disease development if there had already been development of the disorder or disease.

The term “prodrug” refers to a drug or compound (active principal) that elicits the pharmacological action resulting from conversion by metabolic processes within the body. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase the effective water solubility of the therapeutic compound for targeting to regions where water is the principal solvent. For example, Fedorak, et al., Am. J. Physiol, 269:G210-218 (1995), describe dexamethasone-beta-D-glucuronide. McLoed, et al., Gastroenterol., 106:405-413 (1994), describe dexamethasone-succinate-dextrans. Hochhaus, et al., Biomed. Chrom., 6:283-286 (1992), describe dexamethasone-21-sulphobenzoate sodium and dexamethasone-21-isonicotinate. Additionally, J. Larsen and H. Bundgaard, Int. J. Pharmaceutics, 37, 87 (1987) describe the evaluation of N-acylsulfonamides as potential prodrug derivatives. J. Larsen et al., Int. J. Pharmaceutics, 47, 103 (1988) describe the evaluation of N-methylsulfonamides as potential prodrug derivatives. Prodrugs are also described in, for example, Sinkula et al., J. Pharm. Sci., 64:181-210 (1975).

The term “treat” or “treatment” as used herein refers to any treatment of a disorder or disease associated with a cortisol-mediated disease or disorder, in a subject, and includes, but is not limited to, preventing the disorder or disease from occurring in a subject who may be predisposed to the disorder or disease, but has not yet been diagnosed as having the disorder or disease; inhibiting the disorder or disease, for example, arresting the development of the disorder or disease; relieving the disorder or disease, for example, causing regression of the disorder or disease; or relieving the condition caused by the disease or disorder, for example, stopping the symptoms of the disease or disorder.

The following definitions are used, unless otherwise described halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine anti-infectious activity using the standard tests described herein, or using other similar tests which are well known in the art.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; heterocycloalkyl and heterocycloalkylalkyl includes the foregoing cycloalkyl wherein the ring optionally comprises 1-2 S, non-peroxide O or N(R⁸) as well as 2-5 carbon atoms; such as morpholinyl, piperidinyl, piperazinyl, indanyl, 1,3-dithian-2-yl, and the like; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be formyl, acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be alkyl substituted with 1 or 2 OH groups, such as hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 3,4-dihydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), 1H-indolyl, isoquinolyl (or its N-oxide) or quinolyl (or its N-oxide).

The term “retrovirus” includes, but is not limited to, the members of the family retroviridae, including alpharetroviruses (e.g., avian leukosis virus), betaretroviruses (e.g., mouse mammary tumor virus), gammaretroviruses (e.g., murine leukemia virus), deltaretroviruses (e.g., bovine leukemia virus), epsilonretroviruses (e.g., Walley dermal sarcoma virus), lentiviruses (e.g., HIV-1) and spumaviruses (e.g., human spumavirus).

The compounds of formula (I) wherein Y and Z are ═O (oxo), are formally N-phenacyl derivatives of heterocyclic- or heteroaryl-alpha-amino acid piperazinyl amides. Thus, methods generally applicable to peptide synthesis can be employed to prepare compounds of formula I. For example, see published PCT application WO 02/094857, U.S. Pat. Nos. 6,043,218, 6,407,211 and 5,583,108.

In general, compounds of formula (I) wherein Ar is 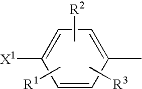
wherein X¹, R¹, R², R³, R⁴, R⁵, Het n and p are as defined above and X and Y are ═O are prepared from aminoalkyl derivatives of formula II as shown in Scheme 1, below, wherein L is Cl or Br. 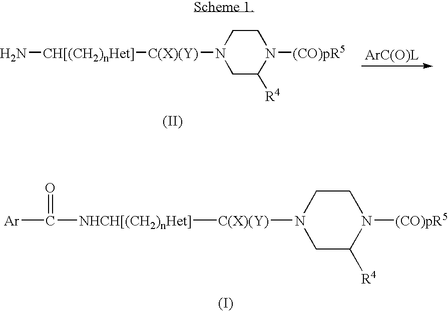
Preparation of Compounds of Formula II.

A compound of formula IIa, is prepared as shown in Scheme 2, below. 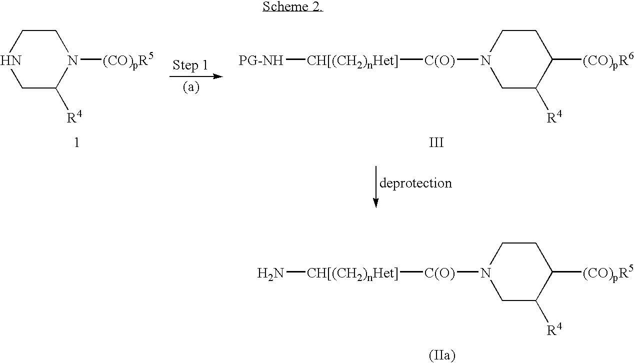
In general, compounds of formula IIa, are prepared in two steps by first converting a compound of formula I to an N-protected aminoalkyl derivative of formula III via methods (a), followed by removal of the amino protecting in III, as described below.
Preparation of Compounds of Formula III
Method (a)

In method (a), an N-protected aminoalkyl derivative of formula III where PG is an amino protecting group (e.g., tert-butoxycarboyl (BOC), benzyloxycarbonyl (CBZ), benzyl, and the like) is prepared by reacting a compound of formula 1 with a compound of formula 4:
PG-NH—CH[(CH₂)ₙHet]X (4)
where X is carboxy (—COOH) or a reactive carboxy derivative, e.g., acid halide. The reaction conditions employed depend on the nature of the X group. If X is a carboxy group, the reaction is carried out in the presence of a suitable coupling agent (e.g., N,N-dicyclohexylcarbodiimide, 1-(3-dimethylaminopropyl)-3-ethylcarbodiimide, and the like) in a suitable organic solvent (e.g., methylene chloride, tetrahydrofuran, and the like) to give an amide intermediate. If X is an acid derivative such as an acid chloride, the reaction is carried out in the presence of a suitable base such as triethylamine, pyridine in an organic solvent (e.g., methylene chloride, dichloroethane, N,N-dimethylformamide, and the like) to give an amide intermediate.

In general, compounds of formula 4 which are N-protected, heterocyclic or heteroaryl α-amino acids or are derived therefrom, are either commercially available or they can be prepared by methods well known in the field of organic chemistry.

Generally, both natural and unnatural amino acids useful in the present invention are commercially available from vendors such as Sigma-Aldrich and Bachem. Examples of natural amino acids are tryptophan and histidine. Unnatural amino acids include, 3-(indan-3-yl)-2-aminopropanoic acid, 3-(morpholin-1-yl)-2-aminopropanoic acid, 3-(piperidin-1-yl)-2-aminopropanoic acid, 3-(piperazin-1-yl)-2-aminopropanoic acid, 3-(pyridin-2-yl)-2-aminopropanoic acid, 4-(pyridin-2-yl)-2-aminobutanoic acid, 4-(imidazol-2-yl)-2-aminobutanoic acid, 4-(benzofuran-2-yl)-2-aminobutanoic acid; 3-(1,3-dithian-2-yl)-2-aminopropanoic acid and the like:

Compounds of formula 4 where X is an acid derivative, e.g., an acid chloride, can be prepared from the corresponding acids of formula 4 (X is —COOH), by chlorinating the carboxy group with a suitable chlorinating agent (e.g., oxyalyl chloride, thionyl chloride and the like) in a suitable organic solvent such as methylene chloride and the like.

Method (b)

Compounds of formula I are prepared as shown in Scheme C below by reacting a piperazine of formula 7 with a compound of formula 6, followed by the removal of the amino protecting group, utilizing the reaction conditions described in method (a) above. 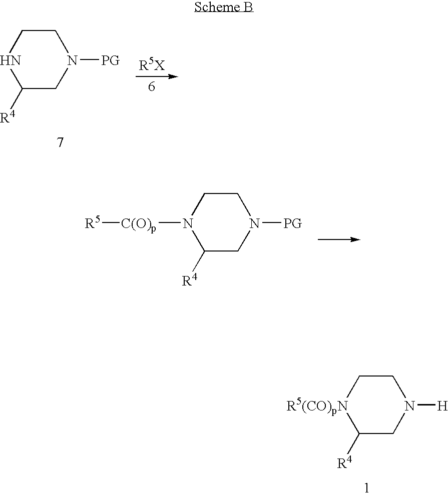
reaction conditions described in method (a) above. Method (b) is particularly suitable for preparing compounds of Formula IIa wherein R⁵X contains an amido or a carbonyl group.

In general, compounds of formula 6 which can also be used to introduce the moiety [X¹(R¹)(R²)(R³)Ph]C(O) into the compound of formula I are commercially available or can be prepared by methods well known in the art. For example, arakyl halides and arakyl acids such as benzyl bromide, 3,4-dichlorobenzyl bromide, phenylacetic acids and 2-phenylpropionic acids are commercially available. Others can be prepared from suitable starting materials such as phenylacetic acid, phenylpropanol, 2-pyridineethanol, nicotinic acid etc., by following procedures described for the synthesis of compounds of formula 4 in method (a) above.

Piperazines and homopiperazines of formula 7 such as piperazine, 2 or 3-methylpiperazines and homopiperazine are commercially available. Piperazines 7 can also be prepared by following the procedures described in the European Pat. Pub. No. 0 068 544 and U.S. Pat. No. 3,267,104.

Compounds of Formula (I) where Ar is 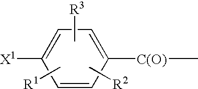
are prepared as described in Scheme C below:

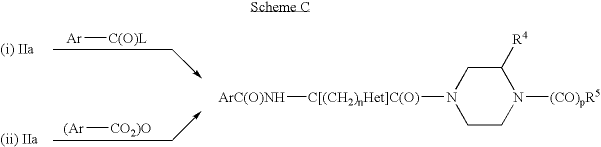

A compound of Formula (I) can be prepared, either:

(i) by reacting a compound of Formula Ia, with an acylating reagent Ar—C(O)L, wherein L is a leaving group under acylating conditions, such as a halo (particularly Cl or Br) or imidazolide. Suitable solvents for the reaction include aprotic polar solvents (e.g., dichloromethane, THF, dioxane and the like). When an acyl halide is used as the acylating agent the reaction is carried out in the presence of a non-nucleophilic organic base (e.g., triethylamine or pyridine, preferably pyridine); or

(ii) by heating a compound of formula Ia with an acid anhydride. Suitable solvents for the reaction are tetrahydrofuran, dioxane and the like; or (iii) reacting a compound of formula IIa, or a compound of formula H₂NCH-((Alk)Het)C(O)Ot-Bu (8) with a compound of formula ArCHO in the presence of NaCNBH₄, followed by hydrolysis of the ester group, if present. Many alpha-amino acid t-butyl esters are commercially available, e.g., from Bachem.

Thus, a specific value for R¹ in formula I, above is H, (C₂-C₄)alkyl, (C₂-C₄)alkoxy or (C₃-C₆)heterocycloalkyl.

A specific value for R2 is H.

A specific value for R3 is H.

A specific value for X¹ is NO₂.

A specific value for N(R⁶)(R⁷) is amino, diethyl amino, dipropylamino, cyclohexylamino, or propylamino.

A specific value for (Alk) is —(CH₂)—.

A specific value for R⁴ is CH₃.

A specific value for R⁵ is cyclopropyl.

Another preferred group of compounds are compounds of formula I which are 4-N-alkanoylpiperazin-1-yl-carbonylalkylbenzamides.

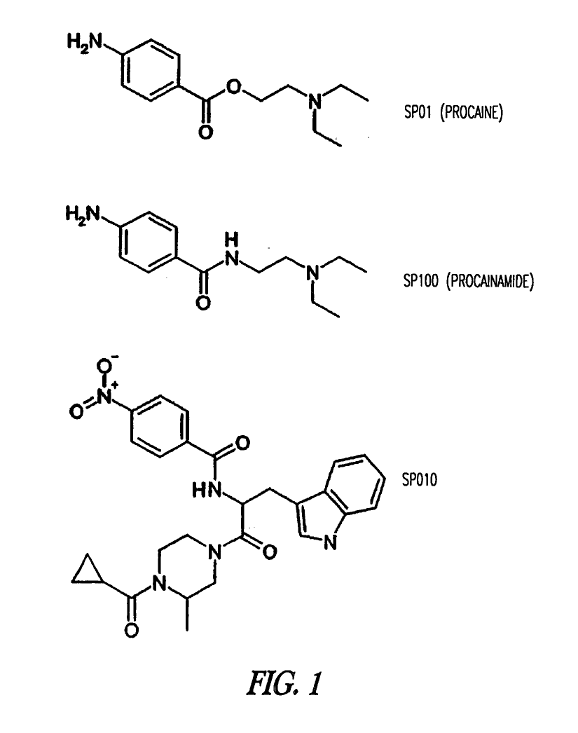

A preferred compound of the invention is SP10 (FIG. 1).

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium), alkaline earth metal (for example calcium or magnesium) or zinc salts can also be made.

Benzoic acid derivatives may be chemically synthesized or derived from plant extracts and may be identified by in silico screeing of chemical and natural product databases. Several procaine derivatives that may be useful in the present invention are listed in Table 1.

TABLE 1

| | Chemical denomination | Origin |
|---|---|---|
| SP010 | 1-(4-cyclopropanecarbonyl-3-methyl- | Chemical |
| | piperazin-1-yl)-2-(1H-indol-3-yl-methyl)- | Synthesis |
| | 4-(4-nitrophenyl)-butane-1,4-dione-3-aza | |
| SP014 | Acetic acid 4,5-diacetoxy-2-acetoxymethyl- | Viburnum |
| | 6-[4-(2-diethylamino-ethylcarbamoyl)- | awabuki |
| | 2-methoxy-phenoxy]-tetrahydro-pyran-3-yl | (Caprifoliaceae) |
| | ester | |
| SP016 | Acetic acid 5-acetoxy-3-(4-benzoyl- | Inula |
| | piperazin-1-yl-methyl)-4-hydroxy-4a,8- | Britanica |
| | dimethyl-2-oxo-dodecahydro-azuleno[6,5-b] | (Asteraceae) |
| | furan-4-yl ester | |
| SP017 | 3-(4-benzoyl-piperazin-1-yl-methyl)-6, | Artemisia |
| | 6a-epoxy-6,9-dimethyl-3a,4,5,6,6a,7,9a,9b- | glabella |
| | octahydro-3H-azuleno[4,5-b]furan-2-one | (Asteraceae |

A compound of the present invention also includes a pharmaceutically-acceptable salt, an ester, an amide, an enantiomer, an isomer, a tautomer, a polymorph, a prodrug, or a derivative thereof. Such salts, for example, can be formed between a positively charged substituent in a compound (e.g., amino) and an anion. Suitable anions include, but are not limited to, chloride, bromide, iodide, sulfate, nitrate, phosphate, citrate, methanesulfonate, trifluoroacetate, and acetate. Likewise, a negatively charged substituent in a compound (e.g., carboxylate) can form a salt with a cation. Pharmaceutically acceptable cations include metallic ions and organic ions. More preferred metallic ions include, but are not limited to appropriate alkali metal (Group Ia) salts, alkaline earth metal (Group IIa) salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like. Examples of prodrugs include esters and other pharmaceutically acceptable derivatives, which, upon administration to a subject, are capable of providing compounds described above.

The compounds of the present invention are usually administered in the form of pharmaceutical compositions. These compositions can be administered by any appropriate route including, but not limited to, oral, nasogastric, rectal, transdermal, parenteral (for example, subcutaneous, intramuscular, intravenous, intramedullary and intradermal injections, or infusion techniques administration), intranasal, transmucosal, implantation, vaginal, topical, buccal, and sublingual. Such preparations may routinely contain buffering agents, preservatives, penetration enhancers, compatible carriers and other therapeutic or non-therapeutic ingredients.

The present invention also includes a pharmaceutical composition that contains the compound of the present invention associated with pharmaceutically acceptable carriers or excipients. In making the compositions of the present invention, the compositions(s) can be mixed with a pharmaceutically acceptable excipient, diluted by the excipient or enclosed within such a carrier, which can be in the form of a capsule, sachet, or other container. The carrier materials that can be employed in making the composition of the present invention are any of those commonly used excipients in pharmaceutics and should be selected on the basis of compatibility with the active drug and the release profile properties of the desired dosage form.

Illustratively, pharmaceutical excipients are chosen below as examples:

(a) Binders such as acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like.

(b) Disintegration agents such as starches, pregelatinized corn starch, pregelatinized starch, celluloses, cross-linked carboxymethylcellulose, sodium starch glycolate, crospovidone, cross-linked polyvinylpyrrolidone, croscarmellose sodium, microcrystalline cellulose, a calcium, a sodium alginate complex, clays, alginates, gums, or sodium starch glycolate, and any disintegration agents used in tablet preparations.

(c) Filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

(d) Surfactants such as sodium lauryl sulfate, sorbitan monooleate, polyoxyethylene sorbitan monooleate, polysorbates, polaxomers, bile salts, glyceryl monostearate, Pluronic™ line (BASF), and the like.

(e) Solubilizer such as citric acid, succinic acid, fumaric acid, malic acid, tartaric acid, maleic acid, glutaric acid sodium bicarbonate and sodium carbonate and the like.

(f) Stabilizers such as any antioxidation agents, buffers, or acids, and the like, can also be utilized.

(g) Lubricants such as magnesium stearate, calcium hydroxide, talc, sodium stearyl fumarate, hydrogenated vegetable oil, stearic acid, glyceryl behapate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like.

(h) Wetting agents such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like.

(i) Diluents such lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like.

(j) Anti-adherents or glidants such as talc, corn starch, DL-leucine, sodium lauryl sulfate, and magnesium, calcium, or sodium stearates, and the like.

(k) Pharmaceutically compatible carrier comprises acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, or pregelatinized starch, and the like.

Additionally, drug formulations are discussed in, for example, Remington's The Science and Practice of Pharmacy (2000). Another discussion of drug formulations can be found in Liberman, H. A. and Lachman, L., Eds., Pharmaceutical Dosage Forms, Marcel Decker, New York, N.Y., 1980.

Besides being useful for human treatment, the present invention is also useful for other subjects including veterinary animals, reptiles, birds, exotic animals and farm animals, including mammals, rodents, and the like. Mammal includes a primate, for example, a monkey, or a lemur, a horse, a dog, a pig, or a cat. A rodent includes a rat, a mouse, a squirrel, or a guinea pig.

Thus, the present compounds may be systemically administered, e.g., orally, in combination with a pharmaceutically acceptable vehicle such as an inert diluent or an assimilable edible carrier. They may be enclosed in hard or soft shell gelatin capsules as powders, pellets or suspensions or may be compressed into tablets. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like or enteric coatings.

A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices, such as patches, infusion pumps or implantable depots.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection, infusion or inhalation can include sterile aqueous solutions or dispersions. Sterile powders can be prepared comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate, cellulose ethers, and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508). The pharmaceutical compositions can be administered in the form of a suppository or the like. Such rectal formulations preferably contain the compound of the present invention in a total amount of, for example, about 0.075 to about 75% w/w, or about 0.2 to about 40% w/w, or about 0.4 to about 15% w/w. Carrier materials such as cocoa butter, theobroma oil, and other oil and polyethylene glycol suppository bases can be used in such compositions. Other carrier materials such as coatings (for example, hydroxypropyl methylcellulose film coating) and disintegrants (for example, croscarmellose sodium and cross-linked povidone) can also be employed if desired.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1-25 wt %, preferably from about 0.5-10 wt %. The concentration in a semi-solid or solid composition such as a gel or a powder will be about 0.1-5 wt %, preferably about 0.5-2.5 wt %.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 mg to as much as 1-3 g, conveniently 10 to 1000 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline. For example, as much as about 0.5-3 g of a compound of formula I can be dissolved in about 125-500 ml of an intravenous solution comprising, e.g., 0.9% NaCl, and about 5-10% glucose. Such solutions can be infused over an extended period of up to several hours, optionally in conjunction with other anti-viral agents, antibiotics, etc. The active ingredient can also be orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a compound of the invention to act as an antiviral agent may be determined using pharmacological models which are well known to the art, or using tests described below.

The following illustrate representative pharmaceutical dosage forms, containing a compound of formula I, for therapeutic or prophylactic use in humans.

| (i) Tablet 1 | mg/tablet |
|---|---|
| SP10 | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| SP10 | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| SP10 | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| SP10 (free base form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| SP10 (free base form) | 10.0 |
| Monobasic sodium phosphate | 0.3 |
| Dibasic sodium phosphate | 1.1 |
| Polyethylene glycol 400 | 200.0 |
| 01 N Sodium hydroxide solution | q.s. |
| (pH adjustment to 7.0-7.5) | |
| Water for injection | q.s. ad 1 mL |

| (vi) Aerosol | mg/can |
|---|---|
| SP10 | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be prepared by conventional procedures well known in the pharmaceutical art.

The invention will be further described by reference to the following detailed examples.

### EXAMPLE 1

### Synthetic Protocol for the Compound SP010

### A. [1-(1H-indol-3-ylmethyl)-2-(3-methyl-piperazin-1-yl)-2-oxo-ethyl]carbamic acid terbutyl ester (B)

Boc-L-Tryptophan (A) (4.556 g; 15 mmol) was dissolved in CH₂Cl₂ (DCM) (60 ml), 1,1′-carbonyldiimidazole (CDI) (2.513 g, 15.5 mmol) was added and then the reaction mixture was stirred at RT for 100 min. 2-Methylpiperazine (1.502 g; 15 mmol) was added and stirring was continued at RT for 6 more hours. 1,2-Dichloroethane (DCE) (15 ml) was added and the organic solution was washed with 5% aq. Na₂CO₃, 3% aq. HCl and water, respectively. The organic phase was dried over Na₂SO₄, filtered and evaporated to dryness. The residue was solidified with diethyl ether-hexane mixture to obtain the title product (B) as a white crystalline solid (3.021 g; 52%).

### B. [2-(4-cyclopropanecarbonyl-3-methyl-piperazin-1-yl)-1-(1H-indol-3-ylmethyl)-2-(3-methyl)-2-oxo-ethyl]carbamic acid terbutyl ester (C)

The piperazine derivative obtained in the previous step (B) (3.021 g; 7.82 mmol) was dissolved in DCE (30 ml). TEA (15.64 mmol; 2.81 ml) was added followed by the addition of cyclopropanecarbonyl chloride (0.77 g; 7.43 mmol; 0.674 ml). The reaction mixture was stirred at RT for 5 hours. The organic solution was extracted with 3% aq. HCl, 3% aq. Na₂CO₃ and with water, respectively. The organic phase was dried over Na₂SO₄, filtered and evaporated to dryness to obtain the desired product as a white solid (D) (3.245 g; 91%).

### C. 2-amino-1-(4-cyclopropanecarbonyl-3-methyl-piperzin-1-yl)-3-(1H-indol-3-yl)-propan-1-one (D)

The Boc-protected amino acid derivative (C) prepared in the previous step (3.254 g; 7.16 mmol) was dissolved in DCM (5 ml). TFA (8 ml) was added while cooling in an ice-water bath. The cooling bath was removed and the reaction mixture was stirred at RT for 5 hours. The mixture was evaporated to dryness, then 10% aq. NaOH (20 ml) was added to the residue while cooling in an ice-water bath. The aqueous solution was extracted with DCE (2×30 ml) and then the combined organic phase was washed with water to neutrality. The organic solution was dried over Na₂SO₄, filtered and evaporated to dryness to obtain the free amine as a light yellow solid (D) (0.787 g; 32%).

### D. N-[2-(4-cyclopropanecarbonyl-3-methyl-piperazin-1-yl)-1-(1H-indol-3-yl-methyl)-2-oxo-ethyl]-4-nitro-benzamide (SP010)

The amino-compound obtained in the previous step (D) (0.763 g; 1.62 mmol) was dissolved in DCE (30 ml), TEA (4.05 mmol; 0.565 ml) was added followed by the addition of 4-nitrobenzoyl chloride (0.256 g; 1.54 mmol). The reaction mixture was stirred at RT for 5 hours. The organic solution was extracted with 3% aq. HCL, 3% aq. Na₂CO₃ and water respectively. The organic phase was dried over Na₂SO₄, filtered and evaporated to dryness to obtain the desired product as a yellow solid (SP010) (0.79 g; 96%). The progress of every transformation reaction was checked by TLC. The identity and the purity of the final product of each step was qualified and quantified by ¹H-NMR and LC-MS spectroscopy.

### EXAMPLE 2

### In Vitro Study of the Inhibition of HIV-1 IIIB Replication on HeLa Cells by Procaine and Procaine Derivatives

A. Methods

In order to study the viral replication in vitro, the GenPhar (Mt. Pleasant, S.C.) AV-Finder™-HIV Drug Discovery Assay was used, that consists of two components: (1) a cloned, continuous-passage HeLa cell line containing an HIV-1 tat-activated molecular switch and a Green Fluorescent Protein reporter gene and (2) a recombinant adenovirus (rAd) vector containing the genes for all three of the HIV-1 receptor/co-receptors (CD4, CXCR4, and CCR5) to transduce into HeLa cells and convert them into highly susceptible HIV-1 indicator cells for use in the assay. The indicator cells over-express the HIV-1 receptor genes and are readily infected with any HIV-1 strain or isolate. All HIV-1 strains tested thus far, regardless of co-receptor preference, and all subtypes or clades of HIV-1 will infect these indicator cells. Infected cells fluoresce brightly so that the inhibition of virus replication by potential antiviral drugs can be readily detected and quantified using standard laboratory plate reader technology.

Detector plates are set up at day 1 by adding HeLa cells (3000/well) to the adenovirus AD-3R in DMEM containing CCS in 96-well plates and to incubate at 37° C. under 95% humidity and 5% CO₂ for 2 days. Without pre-medication, at day 3, HIV-1 IIIB (200IP/well) and increasing concentrations of procaine, procainamide (both from Aldrich-Sigma), SP10, or reference compounds (AZT, ddI, 3TC) were added and incubated overnight. At day 4, the medium was replaced by fresh medium containing the corresponding concentration of the compounds of interest. The infectivity was assessed by measuring the fluorescence on each well at day 7 (λₑₘᵢₛ=485 nm; λ_{exc}=520 nm). With 24 hours pre-medication, increasing concentrations of procaine, procainamide, SP10 (FIG. 1) or reference compounds (AZT, ddI, 3TC) were added at day 3 and incubated overnight. At day 4, HIV-1 IIIB (200IP/well) and increasing concentrations of procaine, procainamide, SP10 or reference compounds (AZT, ddI, 3TC) were added and incubated overnight. At day 5, the medium was replaced by fresh medium containing the corresponding concentration of compounds of interest and the infectivity was assessed by measuring the fluorescence on each well at day 8. Results are expressed as percentage of inhibition of the viral replication.

Following the above described cell treatment protocol, the levels of cellular 3-(4,5-dimethylthiazol-2-yl)-2,5-diphenyl tetrazolium bromide (MTT) reduction, a measure of mitochondrial integrity, were determined in order to examine whether the compounds tested were cytotoxic.

Procaine HCl was used either alone dissolved in water (SP01) or in an Anticort-like formulation (SP01A) containing zinc sulfate heptahydrate and ascorbic acid at the ratio of about 26-27 (26.6)/1/1-2 (1.6) (for example 200 mg procaine HCl with 7.5 mg of zinc sulfate heptahydrate and 12.5 mg of ascorbic acid; Xu, J. et al. J Pharmacol. Exper. Ther. 2003 307:1148-1157) (Samaritan Pharmaceuticals).

B. Results

1. Effect on HIV-1 IIB Viral Replication. No Pre-Medication.

The structures of the compounds procaine HCl (SP01), procainamide (SP100) and N-(2-(4-Cyclopropanecarbonyl-3-methyl-piperazin-1-yl)-1-(1H-indol-3-yl-methyl)-2-(oxo)-ethyl]-4-nitro-benzamide (SP10) are shown in FIG. 1. SP10 was obtained from Comgenex (Budapest, Hungary). Compounds were dissolved in water or when indicated in the Anticort-like formulation (SP01A, SP100A, SP10A).

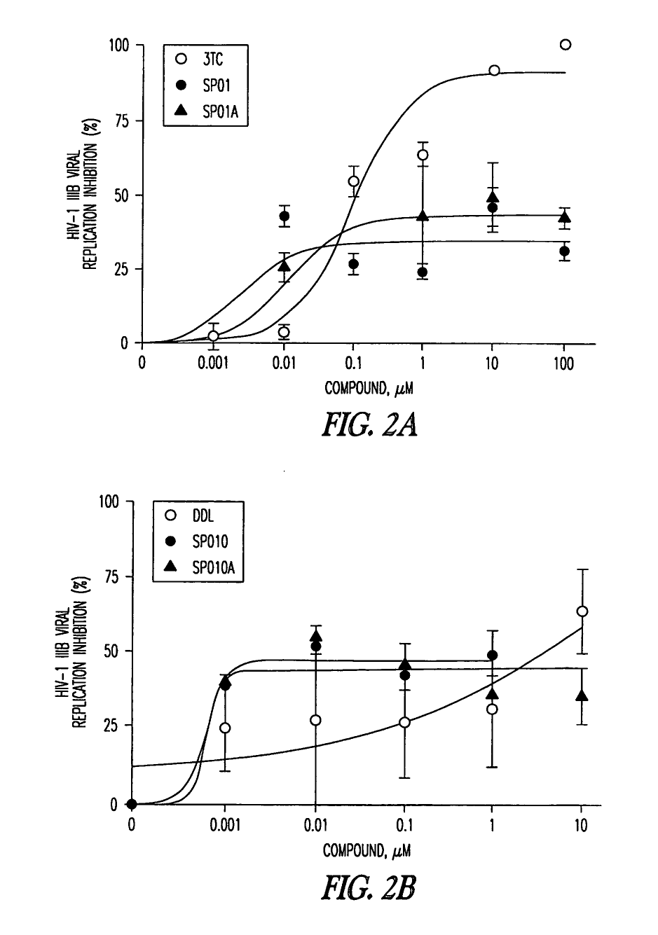

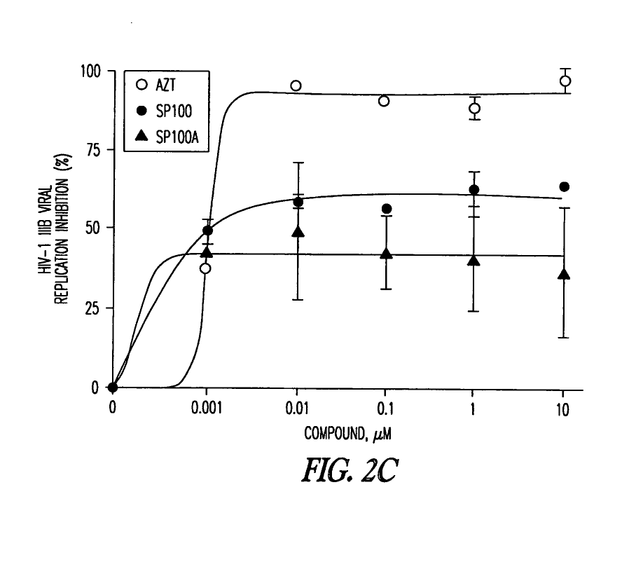

SP01 inhibited the HIV-1 IIIB viral replication with a higher efficacy than the classical antiviral agent 3TC when used at concentrations up to 0.1 μM (FIG. 2A). SP01A also inhibited viral replication in a dose-dependent manner reaching a 43% inhibition compared to 90% inhibition obtained with maximal concentrations of 3TC (FIG. 2A). Interestingly SP01 and SP01A at all concentrations tested, up to 100 μM were devoid of cell toxicity as assessed by the MTT cytotoxicity assay, in contrast to 3TC which showed toxicity with an IC50 of 71 μM. In further studies, the antiviral agents ddI and AZT were found to be cytotoxic with IC50s of 89 and 161 μM concentrations, respectively. Thus, future experiments and in order to be able to accurately compare the antiviral properties of the compounds under investigation to that of classical antiviral agents, concentrations ranging from pM up to 10 μM were used. SP10 and SP10A were found to be more potent that ddI at concentrations up to 1 μM (FIG. 2B), inhibiting viral replication by 40%. For both SP10 and SP10A the strongest inhibition was observed at 0.01 μM inhibiting by 55.60±2.12% and 50.20±1.70% (p>0.001) respectively the viral replication compared to 26.37±26.11% (p<0.05) inhibition observed by ddI.

2. Effect on HIV-1 IIB Viral Replication. Effects of 24 Hours Pre-Medication.

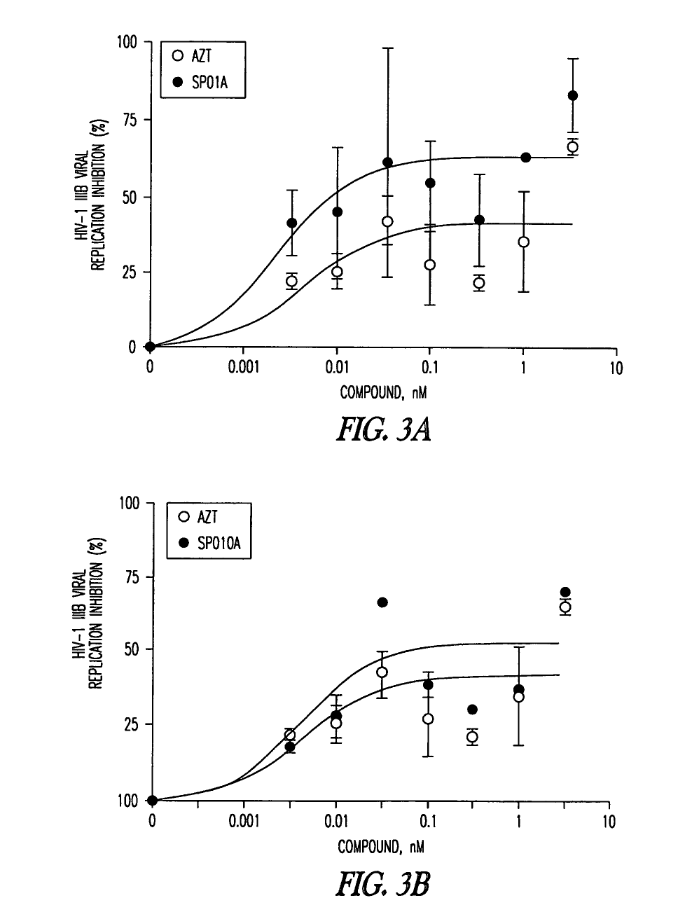

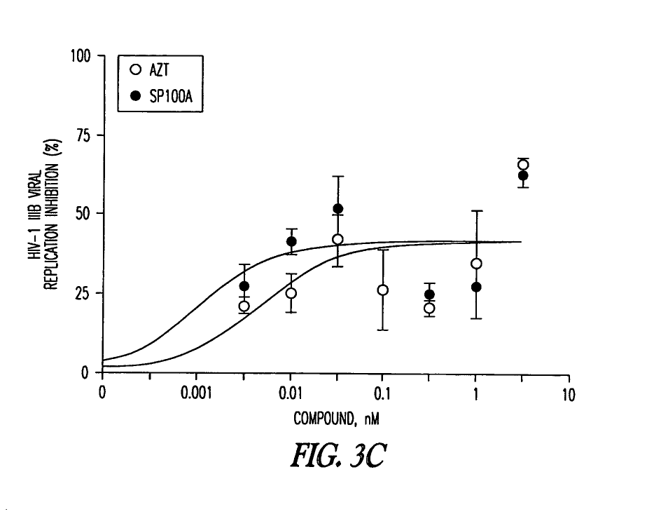

Except for AZT, all the compounds tested were dissolved in the Anticort-like solution. After 24 hours pre-medication, all of them displayed at a concentration or another a better efficacy than AZT on the viral replication (FIG. 3). SP01A (FIG. 3A) and SP010A (FIG. 3B) reduced viral replication in a more dramatic manner compared to AZT reaching a plateau of 63% and 52% inhibition for SP01A and SP10A respectively, compared to 32% inhibition by AZT. The peak of the inhibitory activity observed was 0.03 nM for SP01A and SP010. SP100 was also effective but at the same extent as AZT (FIG. 3C).

3. Effect on HIV-1 IIB Viral Replication. Effects of 48 Hours Pre-Medication.

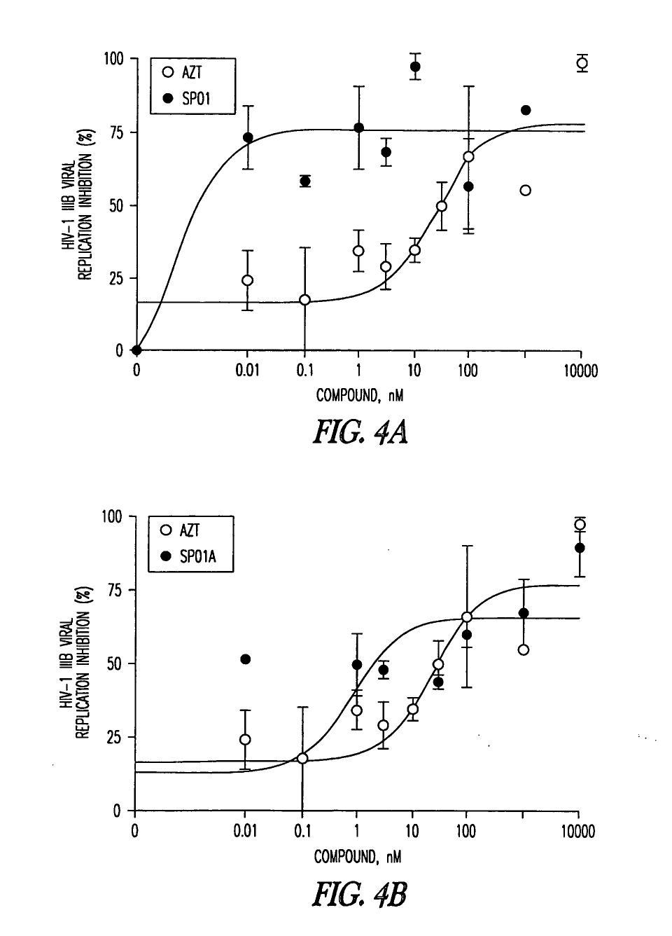

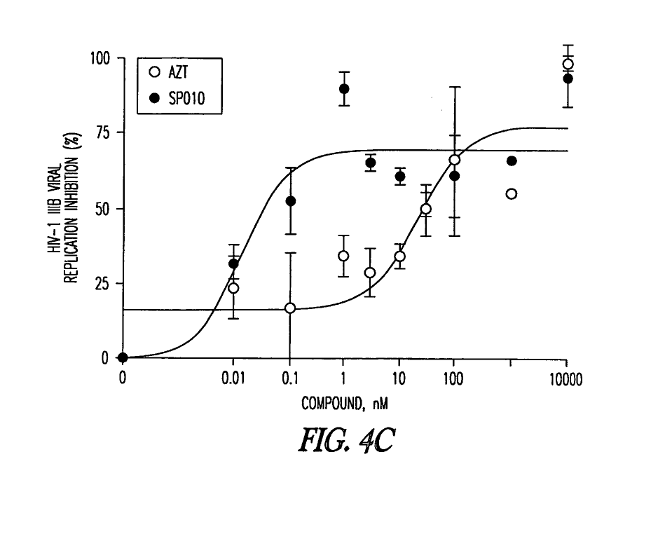

Forty-eight hours pretreatment with SPOI inhibited by 75% HIV replication at all concentrations tested (FIG. 4A). Under the same protocol AZT inhibited the HIV replication in a dose-dependent manner with an IC50 of 30 nM. 48 hours pretreatment with SP01A also inhibited viral replication (FIG. 4B) and the same was true for SP010 which inhibited with an IC50 of 0.01 nM (FIG. 4C).

4. Effect on HIV MDR 769 Viral Replication. Effects Without Pre-Medication.

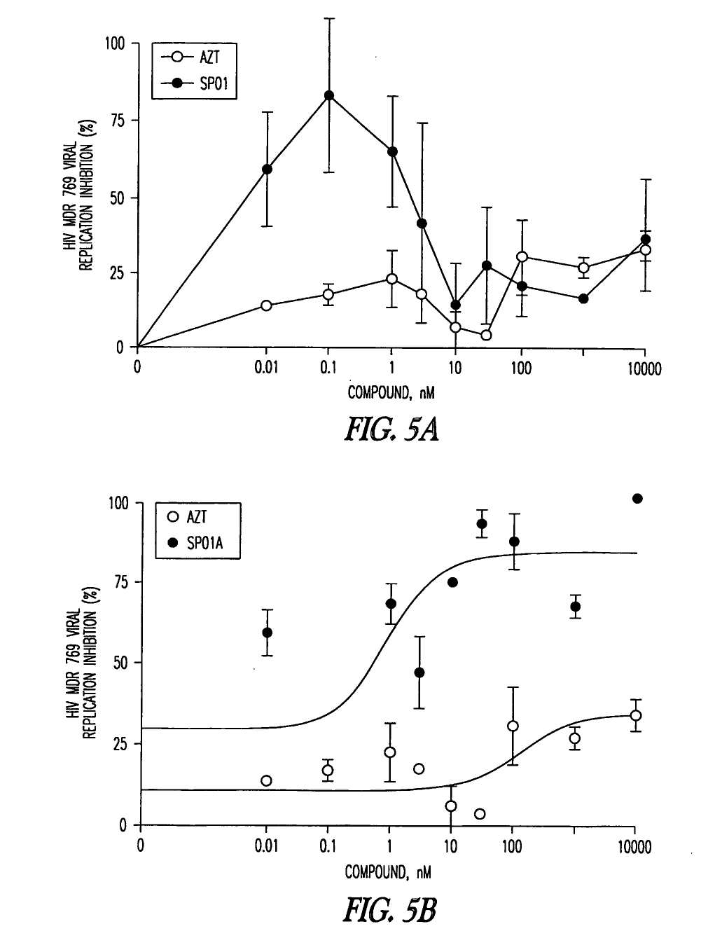

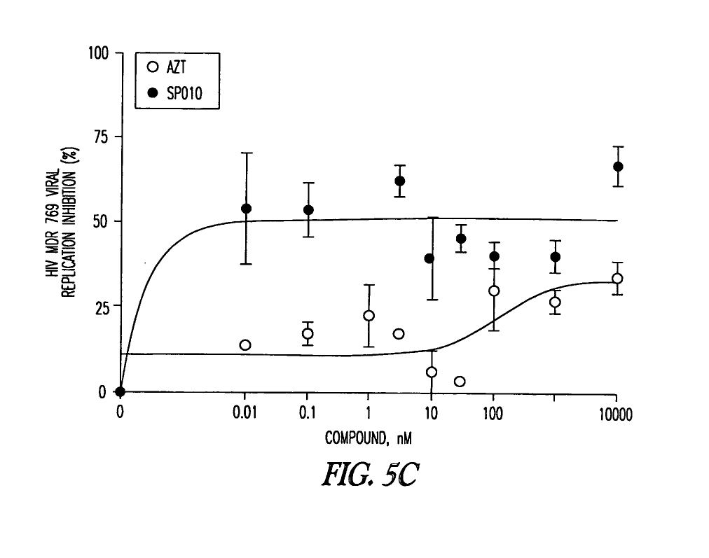

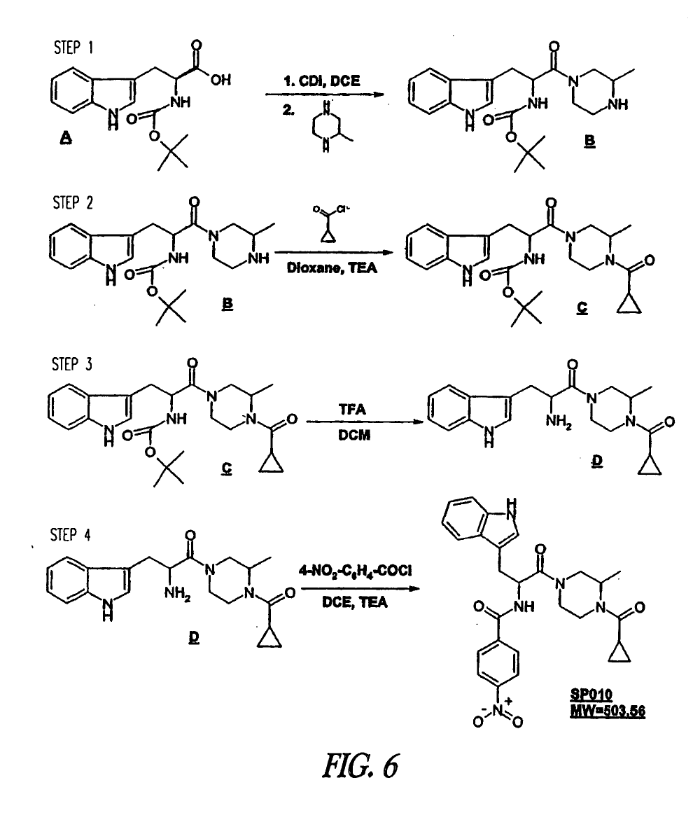

As expected AZT was not effective in inhibiting the HIV MDR 769 strain replication (FIG. 5A,B,C). SP01 inhibited by 75% the HIV MDR 769 viral replication at concentrations up to 1 nM. At higher concentrations the compound was not effective. In contrast SP01A effectively inhibited the MDR HIV strain replication at all concentrations tested, reaching up to 80% inhibition. SP010 also inhibited the replication of the MDR HIV strain although with a maximal efficacy reaching 50%.

### EXAMPLE 3

### Clinical Study

A. Methodology

1. Ethical Conduct of the Study

This study was conducted in accordance with ethical principals that are consistent with good clinical practice and applicable regulatory requirements.

2. Study Drug and Doses Administered

Capsules of 200 mg Procaine HCl were supplied by Samaritan Pharmaceuticals in a formulation containing procaine HCl, zinc sulfate heptahydrate (to decrease the rate of absorption of procaine), ascorbic acid (as an antioxidant), potassium benzoate, and disodium phosphate and sodium sorbate as a preservative. The dose was determined by prior studies of the bioavailability of procaine HCl and the doses used in previous studies of procaine HCl in the treatment of depression in elderly persons (Whalen et al. J. Clin. Epidemiol. 1994 47: 537-546; Cohen et al., Psychosomatics 1974 15: 15-19; Sakalis et al. Current Therapeutic Research 1974 16: 59-63).

3. Selection of Study Population

Eligible patients were ≧18 years, HIV-1 positive (cohorts A, B, C, D); on stable triple antiretroviral regimen for the preceding 8 weeks; with current CD4 counts >200/mm³.

4. Study Design

The study was a non-randomized, Phase II, open-label, single investigative center, eight-week study sequentially using four doses of orally administered procaine HCl: 200 mg (cohort A), 400 mg (cohort B), 600 mg (cohort C) and 800 mg (cohort D). Six subjects were enrolled per cohort. During the screening phase of the study, subjects previously diagnosed with HIV-1 provided written informed consent. Each potential participant underwent complete medical history, and all medications taken within the past 3 months and any current medications were reviewed. Each potential participant underwent clinical laboratory tests, including RNA PCR to determine viral load as well as infection screening (HIV antibody test).

Patients returned on Day 7 to begin the 8 weeks of medication administration. They were given daily medication diaries to record when they are taking their study medication. Subjects underwent complete clinical and biological examinations. HIV negative subjects were discharged, having completed their part of the study. In the subsequent visits of weeks 2, 3, 4, 6, 9 (last dose of medication), each subject underwent clinical laboratory tests, including viral load by NASBA. Patients received their last dose of medication on day 64. Patients returned at week 11 (end of study) for complete laboratory tests.

5. Efficacy Variables: Viral Load Measurements

Viral load was measured by NASBA Assay (Using Nuclisens assay from Organon Technica®) with a lower limit of detection of 50 copies/ml, banked samples were stored at −70° C.

6. Statistical Methods

For each dose level (A-D), changes (week 9—baseline) in efficacy variables were tested for significance using a paired Student t-test (two sided). Analyses of variance (ANOVA) and analyses of covariance (ANCOVA) were conducted to compare the changes in safety and efficacy (covariate=baseline values) variables across the four dose levels, respectively. In addition, regression analyses were conducted to test for a linear trend in efficacy variables across the four dose levels. Changes from baseline to week 9 for all four dose levels combined were tested using paired t-tests. Similar analyses were conducted for changes from week 9 to week 11 to assess potential “rebound effects” after the drug was removed. Mixed effects modeling procedures were used to test for linear and quadratic trends across all study visits. Finally, subgroup analyses which combined low vs. high dose levels were also conducted. The significance level was set at 0.05. Statistical analyses utilized SAS v9.0 (Carey, N.C.).

The results obtained in vitro were analyzed by ANOVA followed by a Dunnett's test.

7. Demographics

30 male patients entered the study, of whom 24 received procaine HCl; there were 12 Caucasian, 7 Hispanic, 9 black, 1 Asian, 1 self-defined as “other.” Mean age was of 42 (38-49) years Cohort A, 46 (39-52) cohort B, 40 (34-60) cohort C and 42 (37-52) cohort D, years. All subjects completed the protocol but one (cohort A) who left the study on day 7 after receiving one dose of study drug and was not replaced.

B. Efficacy Evaluation

1. Viral Load (Table 2)

Because the subjects in the study had to be on HAART, the majority of subjects entered with undetectable viral load measures. But for the patients in the study with detectable viral loads, viral load measures tended to decrease over time. In an attempt to obtain additional measures of viral load changes, stored samples from patients who had undetectable viral loads were run using the more sensitive FDA approved NASBA assay which has a lower limit of detection (50 copies/ml). Results from these assays are shown in Table 2.

TABLE 2
Mean Changed Values Across Cohort and All cohort combined in Viral Load

| | | | | | | | | | | | | | Cohort | Linear |
|---|---|---|---|---|---|---|---|---|---|---|---|---|---|---|
| | Cohort A | | | Cohort B | | | Cohort C | | | Cohort D | | | P- | Trend |
| | Mean | SD | ρ* | Mean | SD | ρ* | Mean | SD | ρ* | Mean | SD | ρ* | value** | P-value |
| A. From Baseline to | | | | | | | | | | | | | | |
| Week 9 | | | | | | | | | | | | | | |
| Viral Load^{†} | −0.52 | 0.98 | 0.30 | −0.21 | 0.65 | 0.51 | −0.79 | 0.42 | 0.03 | −0.54 | 1.46 | 0.41 | 0.23 | 0.78 |
| 2 patients omitted from | −0.64 | 2.15 | 0.60 | 0.48 | 1.49 | 0.51 | −1.82 | 0.97 | 0.03 | −0.10 | 2.10 | 0.92 | 0.40 | 0.87 |
| analysis | | | | | | | | | | | | | | |
| B. From Week 9 to | | | | | | | | | | | | | | |
| Week 11 | | | | | | | | | | | | | | |
| Viral Load^{†} | −0.48 | 0.61 | 0.21 | −0.35 | 0.28 | 0.047 | 0.54 | 1.09 | 0.39 | 0.38 | 0.48 | 0.11 | 0.10 | 0.02 |
| 2 patients omitted from | −1.10 | 1.71 | 0.38 | −0.80 | 0.63 | 0.47 | 1.25 | 2.51 | 0.39 | 1.04 | 1.15 | 0.11 | 0.09 | 0.03 |
| analysis | | | | | | | | | | | | | | |

| | Change values of week 9 from baseline | | | Change values of week 11 from week 9 | | |
|---|---|---|---|---|---|---|
| C. All Cohort combined | Mean | SD | P-value* | Mean | SD | P-value* |
| Viral load PCRI^{†} | −0.50 | 0.96 | 0.03 | 0.04 | 0.73 | 0.81 |
| With 2 patients omitted from analysis | −0.71 | 1.72 | 0.10 | 0.17 | 1.76 | 0.69 |
| Viral load PCR II^{†} | −0.51 | 0.83 | 0.03 | 0.09 | 0.79 | 0.62 |
| With 2 patients omitted from analysis | −0.72 | 1.28 | 0.01 | 0.31 | 1.89 | 0.51 |

^{†}Log transformed Polymerase Chain reaction values, PCRI = all measures; PCRII = only viral load less than 400 copies/ml;
*Two-sided paired t-test;
**Ancova: adjusted for baseline value.

The results are presented using two approaches: first all measurements obtained by the more sensitive assay were used, even if they were over 400, and second, a second analysis was performed using only values from the more sensitive assay, if the new value was less than 400. Analysis of data from the more sensitive assays revealed no significant differences across treatment groups (p=0.23 for update I, and p=0.10 for update II), as well as no significant linear trend across dose levels (p=0.78 for update I and p=0.44 for update II). All four groups exhibited decreases in mean viral load. Comparison of mean changes from week 9 to week 11 (i.e., the post drug administration period), showed that there was a rebound effect seen at the two higher dose groups (C and D) using the more sensitive assay as noted by the significant linear trend (p=0.02 for update I, p=0.01 for update II, Table 2b). As shown in Table 2c, which compares mean changes for all dose groups combined, there was a statistically significant decrease in mean viral load using the more sensitive assays (p=0.03 for update I, p=0.01 for update II). The original viral load measures also showed a more modest decrease that did not reach statistical significance (p=0.22). No rebound effect was noted (p>0.62 for all three analyses). Because two patients changed their antiretroviral therapy during the study, there were some chances that these two patients contributed excessively to the viral load changes seen. Analyses were redone with these two patients omitted. Again in the baseline to week 9 analysis across doses, most groups had a decrease in viral load. Also, from week 9 to week 11 viral load increased, the greatest increase being in the highest doses groups. In conclusion there was a reduction of viral load of about one half log in all groups in the baseline to week 9 analysis. Interruption of drug treatment resulted in a rebound at the two higher doses.

C. Discussion

Procaine (SP01). Procainamide (SP100) and SP010 reduce HIV-1 IIIB replication in human cells with an efficacy higher than AZT, ddI or 3TC. In an experimental protocol without pre-medication, an inhibition of HIV-1 IIIB replication by these compounds was observed up to 50% with concentrations in the nanomolar range and there was not a major difference between the compounds dissolved in water compared to those dissolved in the Anticort formulation (SP01A, SP010A and SP100A). Surprisingly, within the range of 1 nM to 1 μM, SP010 displayed a higher efficacy than ddI in inhibiting viral replication.

In order to assess whether the virus was the direct target of the compounds or another mechanism is mediating the effect of these compounds on viral replication, the HeLa cells were pre-medicated for 24 hours with the different compounds in Anticort-like solution before the virus was added. Interestingly, the effect obtained was much stronger than without pre-medication and with concentrations in the picomolar range. The curve plateau was at more than 63% inhibition for SP01A, 52% for SP010A whereas it was around 32% for AZT. SP100A was less effective than AZT. In addition, the anti-viral activity of SP010A peaked up to 65% inhibition of the replication at 30 pM, and below 60% for SP01A whereas at the same concentration the inhibitory effect of AZT did not reach 30%.

Preincubation of the cells with the compounds under investigation for a 48 hours time period had even more pronounced effects, up to 80% inhibition of viral replication, even at picomolar concentrations. This difference in efficacy displayed after pre-medication versus no pre-medication suggests that the compounds under investigation may not directly target the virus but, more likely, modify the sensitivity of the cells to the virus entry, rendering them more resistant. Several observations established that inhibitors of cholesterol synthesis inhibit cell fusion formation induced by HIV-1 (Srivinas et al., AIDS Res Hum retrovir, 1994 10: 1489-1496) and that drugs extracting cholesterol from the cellular membrane exert an anti-HIV effect in vitro (Sarin et al., N Engl J Med, 1985 313: 1289-1290; Liao et al., AIDS Res Hum retrovir, 2001 17: 1009-1019; Maccarrone et al., J Neurochem, 2002 82(6): 1444-1452). In addition, it has been demonstrated that pre-incubation of procaine decreased the cholesterol synthesis rate limiting HMG-CoA mRNA expression induced by hormonal stimulation in mice and human adrenal cells (Xu et al., J Pharmacol Exp Therap, 2003 307:1147-1157).

These data suggest that procaine and procaine based compounds containing or derived from the SP01, SP010 and SP100 compounds reduce the HIV virus replication by modifying the cholesterol content of the cell membrane, rendering it much more difficult, even impossible, for the virus to entry and infect the cell. If this is true then it is believed that, in contrast to the classical anti-viral agents, such AZT, 3TC and ddI, SP01, SP10 and SP100 should be effective in blocking the HIV MDR 769 virus replication, due to reduced infectivity of the cells. Indeed, although AZT was ineffective in blocking HIV MDR 769 virus replication, SP01, SP010 and SP100 effectively blocked the replication of the virus/infectivity of the cells.

In a clinical setting, administration of procaine (SP01) in the Anticort formulation (SP01A) also caused a significant decrease in viral load of about 0.5 log between baseline and study end in patients under HAART therapy. The determination of viral load was made using a more sensitive assay, which compares favorably with many current NRTI medications.

In conclusion, the data herein demonstrates the ability of procaine, procainamide and the benzamide derivative SP010 to provide new anti-retroviral therapy efficaciuous either alone or in combination with HAART and mega HAART therapies. These results suggest that these compounds act most likely on mammalian cells by increasing their resistance to the virus entry rather than acting directly on the virus itself. Although the mechanism of action is not fully understood, an agent that acts on the host cells rather than directly on the virus can lower the rate of emergence of resistant strains and therefore to increase the efficacy of the current anti-retroviral therapies. The addition of oral procaine HCl in the Anticort formulation to the stable triple antiretroviral regimen of HIV+ patients demonstrated a reduction of viral load and an improvement in patient quality of life after just 9 weeks treatment. The finding that procaine in Anticort reduced the viral load in patients under HAART therapy, where viral load is supposed to be maximally suppressed, is in agreement with the in vitro studies presented above and indicates that the family of compounds disclosed in the present invention are beneficial in cases of resistance to triple antiretroviral therapy in HIV+ patients.

Materials and Methods For Examples 4-7

Y1 mouse adrenal tumor cells were obtained from American Type Culture Collection (Manassas, Va.) and MA-10 mouse Leydig tumor cells were given by Dr. Mario Ascoli (University of Iowa, Iowa). Mouse Hepal-6 cells medium were obtained from American Type Culture Collection (Manassas, Va.). UT-1 cells were provided by Dr. J L Goldstein (Sothwestern University, TX). Fetal-bovine lipo-protein deficient serum (FBLPDS) was from Intracel Corporation (Frederick, Md.). F-12K (Kaign's modification of Ham's F-12) and DMEM culture media were purchased from American Type Culture Collection and DMEM/Ham's F-12 medium, horse serum, and fetal bovine serum (FBS) were purchased from InVitrogen Corporation (Carlsbad, Calif.). Antisera used: anti-20-OH-progesterone (Endocrine Sciences, Calabasas, Calif.), anti-progesterone (ICN Pharmaceuticals, Costa Mesa, Calif.), anti-P-450_{scc} (Research Diagnostics Inc., Flanders, N.J.), anti-G3PDH (Trevigen, Inc., Gaithersburg, Md.). ³H-20a-hydroxyprogesterone, ³H-progesterone, ³H-corticosterone and ³H-mevalonolactone were purchased from PerkinElmer Life Sciences Inc. (Boston, Mass.) and ¹⁴C-HMG-CoA was obtained from Amersham Pharmacia Biotech (Buckinghamshire, England). The MTT cell proliferation assay kit was purchased from Trevigen, Inc. (Gaithersburg, Md.), the PepTag assay for nonradioactive detection of PKA kit was purchased from Promega Corporation (Madison, Wis.) and the Varian Bond-Elut NH2 columns were obtained from Chrom Tech, Inc. (Apple Valley, Minn.). Procaine chlorhydrate and compactin were obtained from chemicals were from Sigma (St. Louis, Mo.). A pharmaceutical composition comprising procaine hydrochloride, zinc sulfate heptahydrate (used to decrease the rate of absorption of procaine), ascorbic acid (used as an antioxidant), potassium benzoate (used as preservative), and disodium phosphate (“procaine-based formulation”) and a placebo of similar composition but devoid of procaine were obtained from Samaritan Pharmaceuticals, Inc. (Las Vegas, Nev.). RNA STAT-60 was from TEL-TEST, Inc. (Friendswood, Tex.). TaqMan® Reverse Transcription Reagents, random hexamers, and SYBR® G Green PCR Master Mix were from Applied Biosystems (Foster City, Calif.). Cells culture supplies were purchased form GIBCO (Grand Island, N.Y.) and cell culture plasticware was from Corning (Corning, N.Y.). All other chemicals used were of analytical grade and were obtained from various commercial sources.

In Silico Screening for Procaine Derivatives

The Interbioscreen (Moscow, Russia) and Comgenex (Budapest, Hungary) databases of chemically synthesized and naturally occurring entities were screened for compounds containing structural homology with procaine using the ISIS software (Information Systems, Inc., San Leandro, Calif.). Selected compounds were screened for their ability to inhibit dbcAMP-induced steroid formation. The structure of the selected biologically active compounds, procaine and derivatives (SP010-014-016-017), are shown in FIG. 7 and the denomination, chemical name and origin for each of these compounds is shown in Table 1.

Animal Treatment

Male 80-day-old Sprague-Dawley rats were purchased form Charles River Breeding Laboratories (Wilmington, Mass.). Rats were housed at the Georgetown University Research Resources Facility under controlled light and temperature, with free access to rat chow and water. They were housed in groups of three and acclimated to their new conditions for 2 days before treatment. All experimental protocols were reviewed and approved by the Georgetown University animal care and use committee. The procaine-based formulation and placebo (both prepared by the University of Iowa School of Pharmacy, Iowa), were administered by gavage in 1 ml volume every day for a total of 8 days. Rats were sacrificed 24 hours later. Corticosterone was measured in organic extracts (ethylacetate/ether, 1:1, v/v) of the collected sera by radioimmunoassay (Amri et al., 1996) under conditions suggested by the supplier of the antisera, ICN Pharmaceuticals (Orangeburg, N.Y.).

Cell Culture

Y1 mouse adrenal tumor cells were cultured in F12K medium containing 15% horse serum, 2.5% FBS and under 5% CO₂ (Brown et al., 1992). MA-10 mouse Leydig tumor cells were cultured in DMEM/F12 medium supplemented with 5% FBS, 2.5% horse serum and under 4% CO₂ (Brown et al., 1992). Human adrenal tumor H295R cells were maintained in DMEM/F12 with 1% ITS⁺ [insulin (1 μg/ml), transferrin (1 μg/ml), selenium (1 μg/ml), linoeic acid (1 μg/ml), and BSA (1.25 mg/ml)], 2.5% Nuserum and 1% Penicillin-Streptomycin at 37° C., 6% CO₂ (Amri et al., 1996). Hepal-6 mouse hepatoma cells were cultured in DMEM medium supplemented with 10% FBS and UT-1 cells were cultured in DMEM/F12 medium supplemented with 8% FBLPDS and 2% FBS plus 40_M Compactin (Chin et al., 1982).

Determination of Steroid Synthesis and Pathway Characterization

Y1 or MA-10 cells were cultured in 96-well plates (2×10⁴ cells per well) for 18 hours, and then treated with increasing concentrations of procaine HCl, a procaine-based formulation or procaine derivatives (SP compounds) for 48 hours. Culture media were then changed and cells were stimulated with 1 mM dbcAMP and the treatment for 24 to 48 hours. To assess cytochrome P-450_{scc} activity and gene expression, culture media were then changed and cells were stimulated with 1 mM dbcAMP and incubated with procaine and/or 22R-hydroxycholesterol 10 mM for another 48 h period. To assess the role of the HMG-CoA reductase activity, culture media were then changed and cells were stimulated with 1 mM dbcAMP and incubated with procaine and/or mevalonate 10 mM for another 48 hour period. The synthesis of 20 —OH progesterone and progesterone in Y1 and MA-10 cell media respectively were measured by RIA (Brown et al., 1992). H295R human adrenal tumor cells were seeded in 48-well plates at 105 cells/well and incubated for 24 hours. After removal of culture media, cells were incubated in the presence of procaine or a procaine-based formulation for another 48 hour-period. At the end of the incubation time period, cells were treated with 1 mM dbcAMP for 48 hours. Cortisol levels in the media were determined by radioimmunoassay as previously described (Amri et al., 1996).

Analysis of Mitochondrial Integrity/Cell Viability

Cell viability at the end of the incubation protocol described above was assessed using the mitochondrial integrity 3-(4,5-dimethylthiazol-2-yl)-2,5-diphenyl tetrazolium bromide (MTT) assay (Trevigen, Gaithersburg, Md.). Briefly, 10 l of the MTT solution were added to the cells in 100 l medium. After an incubation period of 4 hours, 100 l of detergent were added and cells were incubated overnight at 37° C. Formazan blue formation was quantified at 600 nm and 690 nm using the Victor quantitative detection spectrophotometer (EGG-Wallac, Gaithersburg, Md.) and the results expressed as (OD₆₀₀-OD₆₉₀).

PKA Activity Measurement

Y1 cells were cultured in 6-well plates (2×10⁵ cells per well) and treated as described above for steroid biosynthesis. At the end of the incubation, cells were washed twice with PBS and proteins were extracted using an extraction buffer (25 mM tris-HCl, pH 7.4, 0.5 mM EDTA, 0.5 mM EGTA, 10 mM -mercaptoethanol, 0.5 mM PMSF, 1 g/ml leupeptin, and 1 g/ml aprotinin). After centrifugation at 18,500 g for 15 minutes, the supernatants were kept for PKA activity assay. Samples were processed using the PepTag assay for non-radioactive detection of PKA activity following the manufacturer's recommendations (Promega Corporation, Madison, Wis.).

Immunoblotting

At the end of the treatment protocol described above, Y1 cells at 90% confluency were washed 2 times with PBS, sonicated 15 seconds in extraction buffer and centrifuged at 18,500 g for 15 minutes at 4° C. Pellets were resuspended in ice-cold lysis buffer (1% Nonidet 40 in extraction buffer), sonicated briefly, and incubated on ice for 1 hour. After centrifugation (22,500 g×30 min, 4° C.), the supernatant was mixed in sample buffer 6× (0.27 M SDS, 0.6 M dithiothreitol, 0.18 M bromphenol blue in 7 ml of 0.5 M Tris-HCl, pH 6.8, and 3 ml glycerol) and boiled for 5 minutes. Proteins were subjected to SDS-PAGE (4-20% gradient SDS polyacrylamide gel) and electrophoretically transferred onto nitrocellulose membranes. The transblot sheets were blocked with 5% non fat dry milk in 25 mM Tris HCl, pH 7.5, 150 mM NaCl, and 0.1% Tween 20 overnight at 4° C. Membranes were then incubated with appropriately diluted primary antibodies 1:800 for anti-P-450_{scc} (Research Diagnostics Inc. Flanders, N.J.) and 1:200 for anti-StAR (Amri et al., 1996), and the reaction was detected by a peroxidase-conjugated secondary antibody (Santa Cruz Biotechnology, Santa Cruz, Calif.) and enhanced chemiluminescence (Amersham Life, Arlington Heights, Ill.). The densities of the appropriate bands were determined using the OptiQuant Acquisition & Analysis software (Packard BioScience).

PBR Radioligand Binding Assays

Radioligand binding assays were performed as previously described (Papadopoulos et al., 1990, J. Biol. Chem. 265, 3772-3779). In brief, cells were scrapped from culture flasks into PBS, dispersed by trituration, and centrifuged at 1200×g for 5 min. The cells were resuspended in PBS to a final concentration of 10-50 μg protein/100 μl. Saturation binding studies were performed in a final volume of 300 μl in the presence of the radioligand [³H]PK 11195 (specific activity 83.5 Ci/mmol; NEN Life Science Products) at the indicated concentrations. Nonspecific binding was determined in the presence of 6 μM of the homologous non-radioactive ligand. After 180 min incubation at 4° C., the assays were stopped by filtration through Whatman GF/C filters (Clifton, N.J.) and washed with 10 ml PBS. Bound radioactivity was determined by liquid scintillation counting. Bound [³H]PK 11195 and [³H]cholesterol were quantified by liquid scintillation spectrometry. Dissociation constants (Kd), the number of binding sites (Bmax) and Hill coefficients (nH) for PK 11195 and cholesterol were determined by Curve-Fit (Prism version 3.0, GraphPad Software Inc., San Diego, Calif.).

HMG-CoA Reductase Assay

Y1 cells in 12-well plates (1×10⁵ cells per well) were treated with increasing concentrations of procaine HCl for 48 hours. Cells were washed twice with ice-cold PBS and incubated with ice-cold assay buffer (0.1M sucrose, 40 mM KH2PO₄, 30 mM EDTA, 50 mM KCl, 5 mM DTT, 0.25% (v/v) of Brij 96, at pH 7.4) on ice for 20 minutes. After centrifugation for 3 minutes at 14000 g (4° C.) the supernatants were collected and used for HMG-CoA reductase activity assay. The total 150 l assay mixture contained 100-200 g protein and the NADPH-generating system (2.5 mM NADP, 20 mM glucose 6-phosphate and 20 U/ml glucose 6-phosphate dehydrogenase). The reaction was started by adding substrate (¹⁴C-HMG-CoA, 0.1 Ci) and stopped after 45 minutes by adding 10-1 of HCl 6M. ³H-mevalonolactone was also added to the samples as an extraction recovery marker. After an additional 30 minutes incubation time, to allow complete lactonization of the product, the mixture was centrifuged. The supernatant was applied to Bond-Elut NH₂ column and eluted with 1 ml of toluene/acetone (3:1). The eluate was discarded and further 4 ml of toluene/acetone was applied to the column and collected in a scintillation vial for counting ¹⁴C-mevalonate and ³H-mevalonolactone signals (Berkhout et al., 1990).

In separate experiments cells were disrupted by sonication and then treated with procaine. The direct effect of the treatment on HMG-CoA reductase activity in the homogenates was determined as described above.

Real-time Quantitative PCR (Q-PCR)

Cells cultured in 6-well plates for 18 hours were treated with increasing concentrations of procaine HCl for the indicated time period. After treatment, cells were stimulated with 1 mM dbcAMP for 24 hours. At the end of the incubation, total cell RNA was extracted using RNA STAT-60 (Tel-Test Inc, Friendswood, Tex.) according to the manufacturer's instructions. HMG-CoA reductase mRNA was quantified by Q-PCR using the ABI Prism 7700 sequence detection system (Perkin-Elmer/Applied Biosystems, Foster City, Calif.). RT reaction was performed using TaqMan® Reverse Transcription Reagents with 1 g total RNA and random hexamers as primers for each reaction according to the manufacturer's instructions. For quantifying mouse HMG-CoA reductase mRNA with Q-PCR, the primers were designed according to GenBank Accession Number BC 019782 using PE/AB Primer Express software, which is specifically designed for the selection of primers and probes. The forward primer was 5′-CCAAGGTGGTGAGAGAGGTGTT-3′ (22 nucleotides; SEQ ID NO:1) and reverse primer was 5′-CGTCAACCATAGCTTCCGTAGTT-3′ (23 nucleotides; SEQ ID NO:2), respectively. The primers were synthesized by Bio-Synthesis Inc. (Lewisville, Tex.). Reactions were performed in a reaction mixture consisting of a 20 l volume solution containing 10 l SYBR® Green PCR Master Mix and 1 l primers mix (5 M each) with 2 l cDNA. The cycling conditions were: 15 sec. at 95° C. and 1 min at 60° C. for 40 cycles following an initial step of 2 min at 50° C. and 10 min at 95° C. AmpliTaq Gold polymerase was activated at 95° C. for 10 minutes. The 18S RNA was amplified at the same time and used as an internal control. To exclude the contamination of unspecific PCR products such as primer dimers, a melting curve analysis was applied to all final PCR products after the cycling protocol. Also, the PCR reactions without the RT reaction were performed for each sample in order to exclude genomic DNA contamination. PCR products were collected and run on a 3% (w/v) agarose/TAE gel to confirm the product size. The threshold cycle (Ct) values for 18S RNA and samples were calculated using the PE/AB computer software. Ct was determined at the most exponential phase of the reaction. Relative transcript levels were calculated as x=2^{Ct}, in which Ct=E−C, and E=Ctₑₓₚₑᵣᵢₘₑₙₜ−Ct_{18S}, C=Ct_{control}−Ct₁₈ₛ.

Protein Measurement

Protein was measured using the Bio-Rad protein assay kit (Bio-Rad Laboratories, Hercules, Calif.) and bovine serum albumin as a standard.

Statistics

Statistical analysis was performed by one-way analysis of variance (ANOVA) and unpaired Student's t test using the INSTAT 3.00 package from GraphPad (San Diego, Calif.).

### EXAMPLE 4

### Procaine and Procaine Derivatives Inhibit the dbcAMP-Induced Steroid Formation in Mouse and Human Adrenal Cell Lines

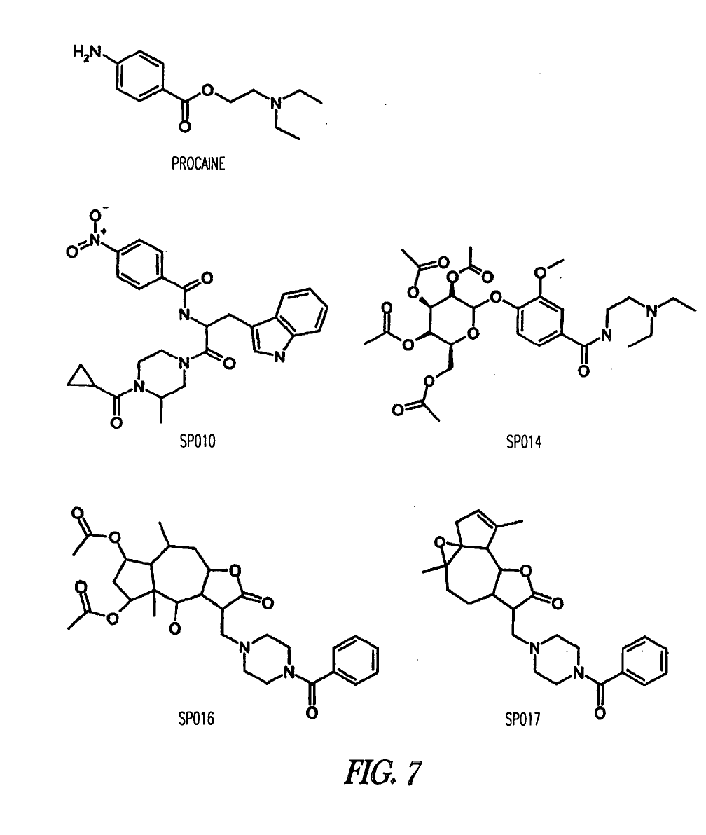

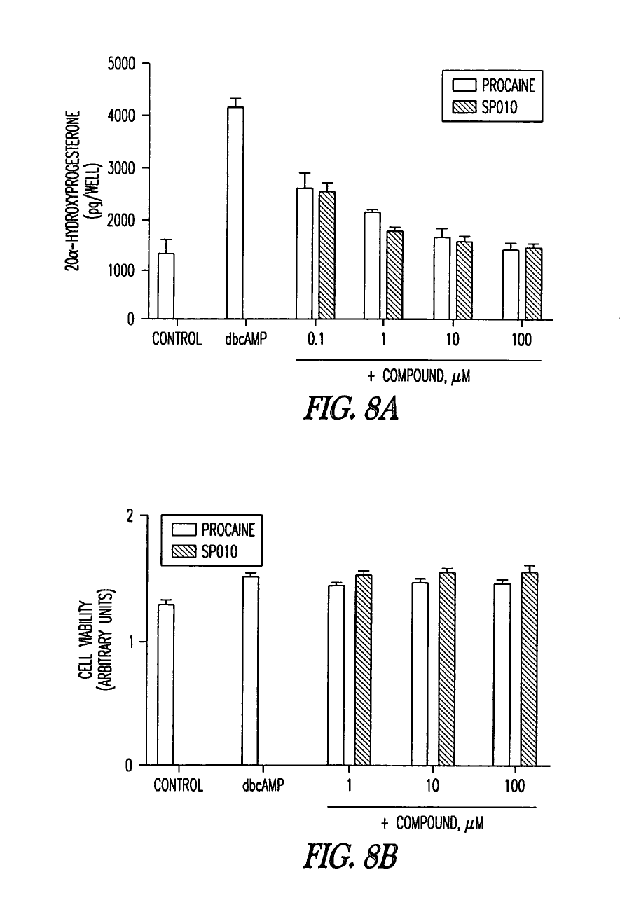

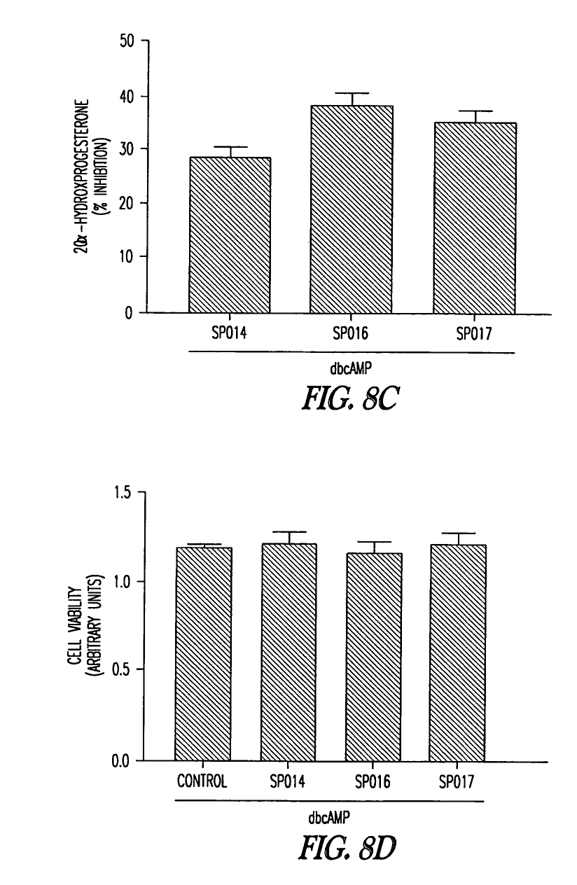

Treatment of Y1 cells with dbcAMP increased 20á-hydroxyprogesterone production by approximately 4-fold (FIG. 8A; p<0.001). Procaine and the procaine derivative SP010 decreased in a dose-dependent manner the dbcAMP-induced 20á-hydroxyprogesterone production (FIG. 8A) following a dose/effect relationship. The procaine derivatives SP014, SP016, and SP017, used at 2 M concentration, reduced the dbcAMP-induced 20á-hydroxyprogesterone synthesis by Y1 cells by 30-38% (FIG. 8C). All compounds tested did not affect basal steroid formation by Y1 cells (data not shown). Moreover, none of the compounds used affected cell viability as determined using the MTT assay (FIGS. 8B & 8D).

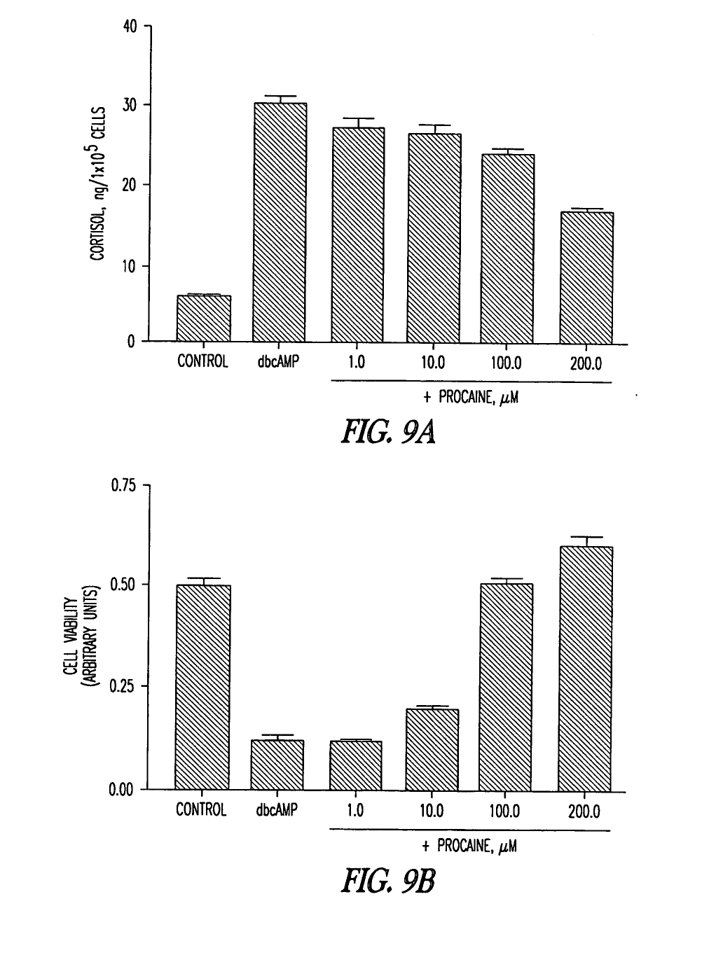

In H295R cells, dbcAMP increased cortisol synthesis by 4-fold (FIG. 9A, p<0.001). Procaine inhibited the dbcAMP-stimulated cortisol production in a dose-dependent manner (p<0.01 by ANOVA) as shown in FIGS. 9A, without effecting basal cortisol production (not shown). Surprisingly, cells exposed to dbcAMP showed a dramatic decrease in cell viability, determined by the MTT assay (FIG. 9B). While not wishing to be bound by theory, cell numbers were not decreased following dbcAMP treatment suggesting that in this case, changes in MTT may reflect mitochondrial function rather than cell viability. Procaine (FIG. 9B) protected against the dbc-AMP-induced change of mitochondrial function.

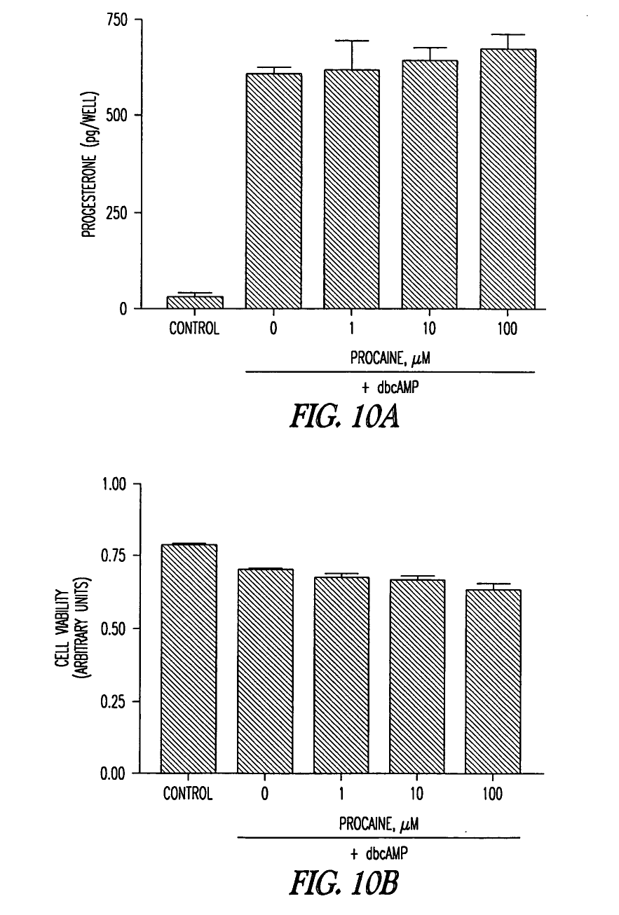

In contrast to adrenal cells, procaine did not affect the dbcAMP-induced progesterone synthesis in MA-10 mouse Leydig tumor cells (FIG. 10A). The treatment did not affect MA-10 cell viability either (FIG. 10B).

### EXAMPLE 5

### Procaine Reduces Circulating Corticosterone Levels in Male Sprague-Dawley Rats

Eight days treatment of adult male rats with a procaine-based formulation reduced serum corticosterone levels by approximately 50% in a significant manner (p<0.05) as assessed by ANOVA (FIG. 11). Similar results were obtained with adult mice (data not shown).

### EXAMPLE 6

### Effect of Procaine on Various Steps of the Steroidogenic Pathway

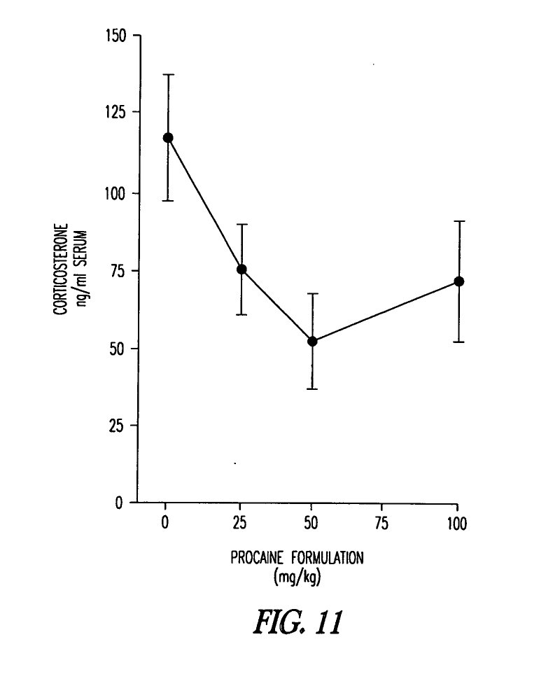

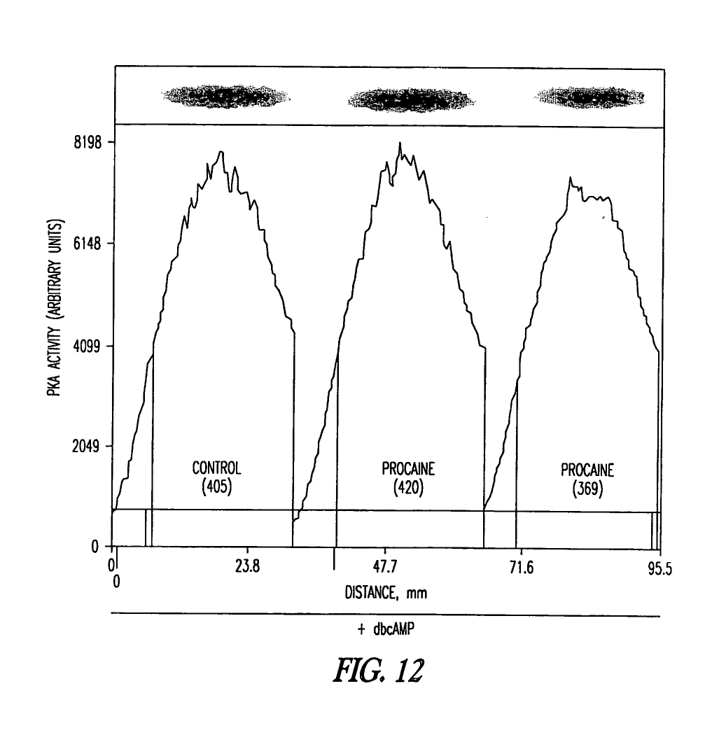

Considering the effect of procaine on the dbcAMP-stimulated steroid formation, the effect of this compound on PKA activity was investigated. PKA activity was measured using a non-radioactive detection kit based on the PKA-specific substrate, PepTag® A1 peptide (L-R-R-A-S-L-G). FIG. 12 shows that procaine at 1 M, which inhibited by 90% the dbcAMP-stimulated steroid formation (FIG. 8A), has no significant effect on the dbcAMP-stimulated PKA activity.

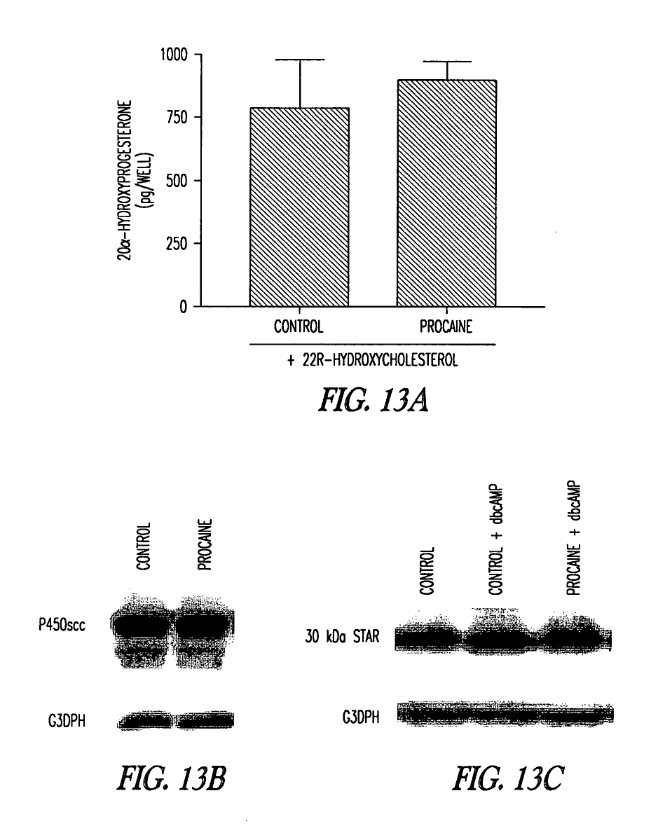

The hydrosoluble cholesterol, substrate of the P-450_{scc}, 22R-hydroxycholesterol induced 7.5-fold increase in 20 —OH progesterone formation, respectively. As shown in FIG. 8A, 1 iM procaine reduced the dbcAMP-induced steroid formation by 90%. However, procaine did not inhibit the effect of 22R-hydroxycholesterol on steroidogenesis (FIG. 13A). In addition, procaine did not modify the expression of the P-450_{scc} enzyme as assessed by immunoblot analysis of cell extracts (FIG. 13B).

While not wishing to be bound by theory, the data presented above indicated that the effect of procaine is beyond the activation of PKA and before cholesterol metabolism to final steroid products. We examined the effect of procaine on two proteins involved in the transport of cholesterol into mitochondria, the peripheral-type benzodiazepine receptor (PBR) and the steroidogenesis acute regulatory protein (StAR) using the same 48 hour treatment protocol with procaine. These experiments showed that 1 μM procaine did not affect either the ligand binding characteristics of PBR (Bmax=27±3 pmol/mg protein and Kd=1.8 nM in control cells vs. Bmax=29±4 pmol/mg protein and Kd=1.7 nM in procaine-treated cells) nor the levels of the mature 30 kDa StAR protein (FIG. 13C) which was induced by 2.5-fold following a 3 hour dbcAMP treatment. Insight of these results, whether cholesterol synthesis itself was affected by procaine was investigated.

### EXAMPLE 7

### Procaine Inhibits the HMG-CoA Reductase Activity and mRNA Expression

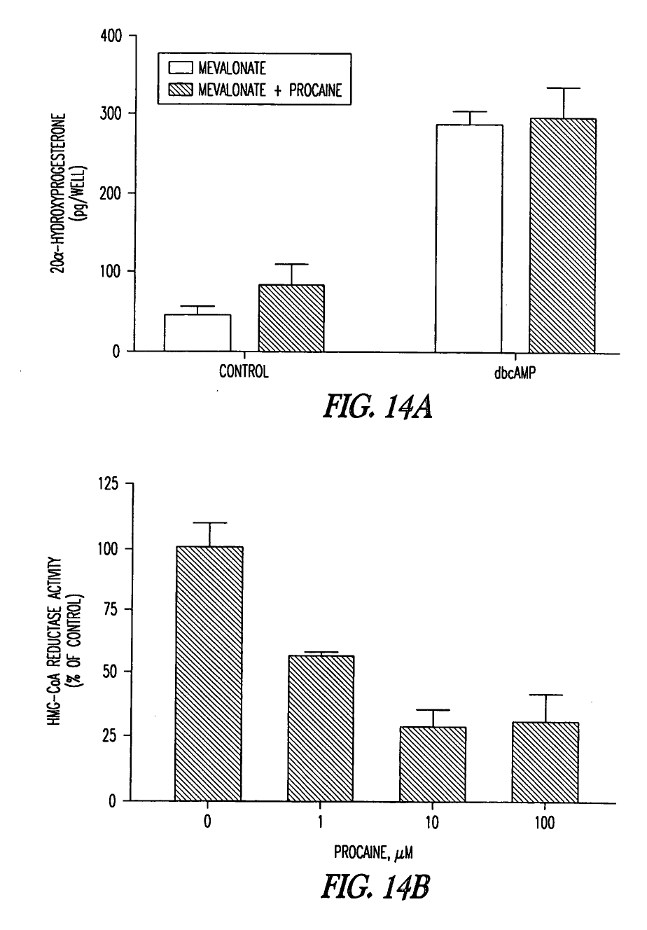

FIG. 14A shows that 1 M procaine did not inhibit the dbcAMP and mevalonate-supported 20á-hydroxyprogesterone formation, indicating that procaine may act at the level of mevalonate synthesis by the HMG-CoA reductase enzyme. HMG-CoA reductase activity was determined in Y1 cells. Procaine reduced in a dose-dependent manner HMG-CoA reductase activity in these cells (FIG. 14B). The percent inhibition for the concentration 1, 10, and 100 M were 44%, 72% and 70 respectively and the effect of the treatment was highly significant (p<0.001 by ANOVA). To assess whether the effect of procaine is due to a direct effect on the enzyme activity, Y1 cells were sonicated and treated with procaine. No direct effect of procaine on HMG-CoA reductase activity was observed (10.1±0.9 pmol/min/mg protein control versus. 9.9±0.01, 10.3±0.6, and 10.1±0.1 pmol/min/mg protein in the presence of 1, 10 and 100 μM procaine, respectively).

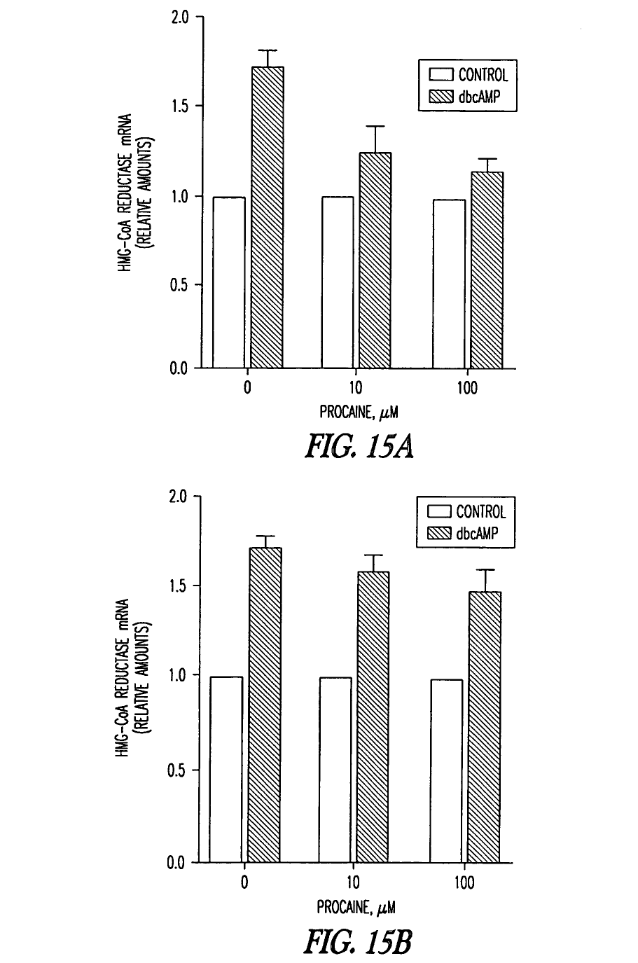

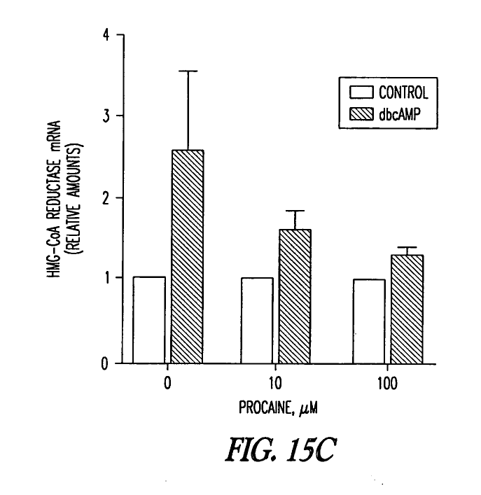

Based on these data we examined the effect of procaine on HMG-CoA reductase mRNA expression levels measured by Q-PCR and using 18SRNA as internal standard. Treatment with dbcAMP for 24 hours induced by 1.8-fold the HMG-CoA reductase mRNA expression (FIGS. 15A). Pretreatment of the cells for 24 hours with procaine reduced in a dose-dependent manner HMG-CoA reductase mRNA levels (p<0.01 by ANOVA) bringing them close to the basal levels (FIG. 15A). Detailed time-course studies indicated that a 6 hour treatment with procaine was the earliest time point when the compound inhibited the dbcAMP-induced HMG-CoA reductase mRNA expression and that this effect was enhanced when cells were pre-treated for 24 hours with procaine (data not shown). Although a trend of inhibition of HMG-CoA reductase mRNA expression was seen in UT-1 cells, a Chinese hamster ovary cell clone containing high levels of HMG-CoA reductase, selected to grow in the presence of compactin, a HMG-CoA reductase inhibitor (Chin et al., 1982), this effect was not significant (FIG. 15B). However, procaine inhibited the dbcAMP-induced HMG-CoA reductase mRNA levels in Hepa1-6 mouse liver hepatoma cells (FIG. 15C) in a significant manner (p<0.01 by ANOVA).

Discussion

Procaine and procaine derivatives modulate the hormone-stimulated corticosteroid formation by adrenal cells in vitro and in vivo by, in one mechanism, reducing the levels of the rate limiting enzyme HMG-CoA reductase mRNA, leading to reduced activity, and decreased cholesterol and corticosteroid biosynthesis.

Y1 mouse adrenal tumor cells have been extensively used to understand the mechanisms underlying adrenal steroid formation. In these cells, 20-hydroxy-progesterone, an intermediate of the steroids synthesis resulting from the conversion of progesterone by 20-hydroxylase, has been used as the steroidogenic index of the cells (Mrotek and Hall, 1977; lida et al, 1989; Brown et al., 1992). As discovered in the present invention, procaine inhibits the cAMP-induced 20-hydroxy-progesterone increase in Y1 cells without affecting basal 20-hydroxy-progesterone production by the cells. Procaine inhibits the cAMP-induced steroid synthesis at concentrations as low as 0.1 μM, and this inhibition displays a dose-response relationship over a wide-range of concentrations. This modulatory effect of procaine on the cAMP-induced steroid formation is not restricted to mouse Y1 cells but is also observed on the H295R human adrenal tumor cells, which synthesize cortisol as the main steroid product. The human adrenal tumor cells are less sensitive to procaine than the mouse adrenal cells. These results confirm and extend previous observations reporting that procaine lowered the steroidogenic effect of a cholinergic muscarinic stimulation (Hadjian et al., 1982) and of dbcAMP (Noguchi et al., 1990) on bovine adrenocortical cells. While not wishing to be bound by theory, these data together with the finding that procaine does not affect basal steroid formation by the cells evidences that procaine exerts its modulatory activity only in the presence of a stimulus.

None of the compounds tested affected adrenal cell viability, determined using the MTT assay. In contrast, in human adrenal tumor cells the treatment with dbcAMP induced a decrease in MTT levels, indicating either an effect on cell viability or an effect of the nucleotide analogue on mitochondrial diaphorase activity. This effect was not seen with Y1 cells and may be specific to H295R cells. Treatment with procaine reversed the effect of dbcAMP on mitochondrial function.

The effect of procaine was not restricted in vitro. Treatment of rats and mice for 8 days with a procaine-based formulation decreased serum corticosteroid levels by 60% compared to placebo. Thus, there is enough corticosterone remaining to support the glucocorticoid-dependent functions. 50% of the measured corticosteroid levels may reflect the normal “unstressed” condition. As the rats have not been pre-conditioned, the stress induced by being handled may be responsible for the stimulation of the corticosterone synthesis and in turn, for an increase of the plasmatic concentrations of this steroid (Kant et al., 1989). Surveys of the literature for circulating corticosterone levels in rats reveals a large variation in the reported values ranging from 4 to 40 ng/ml. Thus, in vivo treatment with procaine does not affect the basal “unstressed” adrenal function but controls the stress-induced glucocorticoid levels, thus maintaining lower “normal” circulating corticosterone levels. Procaine has been also described to decrease the release of corticotropin-releasing factor previously induced in a model of cerebral hemorrhage in rats (Plotsky et al., 1984) and to decrease the release of ACTH in a model of surgically-induced stress in the dog (Ganong et al., 1976). Such a central effect of procaine on hypothalamus and pituitary cannot be excluded to explain the decrease of the corticosterone concentrations observed in the experiments in addition to a direct effect on the adrenal cells, reinforcing the interest of procaine and its derivatives as cortisol-modulating agents.

Because procaine HCl is the ester of diethylaminoethanol and para-aminobenzoic acid and as such it can be easily hydrolyzed in the body, stable and efficient procaine derivatives exhibiting similar properties and no cell toxicity were searched. Thus, procaine derivatives were identified by in silico screening of chemical databases and tested for their ability to modulate the cAMP-corticosteroid formation. From these compounds, SP010 (Table 1) was as potent as procaine even at a concentration as low as 1 μM and displayed the same dose/response effect as procaine, suggesting a common pharmacological mechanism. However, while not wishing to be bound by theory, SP010 may also regulate cortisol levels via a regulation of the intracellular calcium concentration. The raise of the intracellular calcium concentration is a key point in the steroids synthesis-stimulating pathway and procaine has been described to modulate this calcium increase by antagonizing the activity of the ryanodine receptor (Shishan-Barmatz V. and Zchut S. (1994) J. Membr. Biol., 138(1): 103; Zahradnikova A. and Palade P. (1993) Biophys. J., 64(4): 991-1003). As a derivative of procaine, it is legitimate to hypothesize that SP010 exerts the same modulatory effect on the calcium pathway contributing therefore to its modulating activity on the cortisol synthesis. Procaine derivatives may also decrease the cAMP-induced expression of the ryanodine receptor RyR2 mRNA, leading to changes in intracellular calcium levels, thus contributing to its modulating activity on cortisol synthesis. A close look at the dose-response effect of procaine and SP01 on Y1 adrenocortical cells indicates that SP010 is as efficacious as procaine and procaine derivatives and maintained the same efficacy at 1, 10 and 100 iM. No effect of these compounds on basal steroid synthesis and cell viability was seen. These results suggest that the SP compounds identified based on their common procaine chemical motif are other candidates to develop drugs against pathologies due or involving increased activity of the HPA axis and thus high cortisol production.

In search of the mechanism of action of procaine on cAMP-induced adrenal steroidogenesis, the effect on the cAMP-induced PKA activity was researched. Hormone-induced PKA activity initially leads into increased cholesterol transport into mitochondria and later on in increase activity and expression of the P-450_{scc}. The quantification of the dbcAMP-stimulated Y1 cells revealed that treatment with procaine did not affect this enzyme. In addition, procaine did not affect the rate of steroid formation by cells incubated in the presence of 22R-hydroxycholesterol, a cholesterol derivative which can cross freely the mitochondrial membranes and directly load onto the P-⁴⁵⁰_{scc} enzyme as a substrate (Papadopoulos et al., 1990), suggesting that P-450_{scc} and other enzymes involved in the steroidogenic pathway were not affected by the procaine treatment. This result was further supported by the finding that P-450_{scc} enzyme levels were not affected by procaine. Taken together and while not wishing to be bound by theory, these data suggest that procaine and procaine derivatives affect the amount of cholesterol available for steroidogenesis. Such effect may be due either to a change in the rate of cholesterol transfer from intracellular stores into mitochondria or an effect on cholesterol synthesis. Procaine had no effect on the expression levels of PBR and StAR, the two key regulatory proteins mediating the transfer of cholesterol into mitochondria (Papadopoulos, 1998; Stocco, 2000). The finding that addition of the substrate of cholesterol synthesis mevalonate in the media together with dbcAMP resulted in abolishing the inhibitory effect of procaine on the dbcAMP-stimulated steroid formation suggested that procaine's site of action is at a step before mevalonate synthesis.

The rate-limiting enzyme in mevalonate and cholesterol biosynthesis is HMG-CoA reductase. Treatment of the cells with increasing concentrations of procaine followed by stimulation with dbcAMP resulted in the dose-dependent decrease of HMG-CoA reductase activity, assessed by the transformation of ¹⁴C-HMG-CoA into ¹⁴C-mevalonate. Maximal inhibition was achieved in the presence of 10 μM procaine. Considering the absence of a direct effect of procaine on HMG-CoA reductase activity measured in adrenal cell extracts and the fact that the effect was seen following a minimal 6 hour incubation time period, procaine may act on HMG-CoA reductase mRNA levels. Indeed, treatment of Y1 cells with dbcAMP resulted in increased HMG-CoA mRNA levels, in agreement with previous findings that cAMP and hormones regulate HMG-CoA reductase enzyme expression (Ness and Chambers, 2002; Ngo et al., 2002). Procaine inhibited in a dose-dependent manner the dbcAMP-induced HMG-CoA reductase mRNA expression levels, without affecting basal HMG-CoA mRNA levels. This finding is in agreement with the effect of procaine on the cAMP-induced steroid formation. To examine the tissue specificity of the effect of procaine on HMG-CoA mRNA expression two cell types, the UT-1 and Hepa1-6 cells, were used. UT-1 cells is a clone of Chinese hamster ovary cells (CHO-K1) that were selected to grow in the presence of compactin, a competitive inhibitor of HMG-CoA reductase. These cells have a 500-fold higher level of HMG-CoA reductase activity (Faust et al., 1982) and 100- to 1,000-fold more immunoprecipitable HMG-CoA reductase enzyme protein than normal cells (Chin et al., 1982). Hepa1-6 cells is a mouse liver hepatoma clone used because liver is the main organ in cholesterol synthesis. Treatment of both UT-1 and Hepa1-6 cells with dbcAMP induced HMG-CoA mRNA expression. Treatment of the cells with procaine resulted in the dose-dependent decrease of HMG-CoA mRNA levels. This effect was minor and not significant in the UT-1 cells but robust in the Hepa1-6 cells, suggesting that there is a tissue specificity of the effect of procaine on HMG-CoA reductase mRNA expression and activity. The finding that procaine regulates HMG-CoA reductase mRNA levels is a novel observation and the data indicating that liver cholesterol formation might be regulated by procaine is an intriguing finding that might lead to novel therapeutic applications for procaine in the field of hypercholesterolemia and related diseases. Procaine's mechanism of action via the reduction of the cAMP-induced HMG-CoA mRNA levels and SP010's mechanism of action possibly via the regulation of the calcium pathway offer alternative approaches to those currently available for regulating the HMG-CoA reductase activity. Local anesthetics, including procaine, were previously shown to affect sterol biosynthesis at a step beyond mevalonate formation (Bell and Hubert, 1980), most likely by inhibiting the cholesterol esterase (Traynor and Kunze, 1975) and cholesterol acyltransferase (Bell, 1981) enzyme activities. The data does not exclude such actions of procaine or other effects that this molecule might exert at a post-mevalonate step, effects which might be tissue specific as those described on adrenal and liver HMG-CoA reductase enzyme.

Elevated concentrations of cortisol have been reported to be associated with many diseases and to worsen the prognosis. In contrast to the detrimental effects of high levels of cortisol in the pathologies described above, maintenance of the basal cortisol levels is necessary for the maintenance of basic biological functions. Glucocorticoids regulate the metabolism of proteins, carbohydrates and lipids, and are essential to the adaptation to acute physical stressors (Munck et al, 1994). Development of compounds which block the excessive glucocorticoid synthesis without affecting the basal steroid formation has proven to be a difficult task, because it requires the identification of a modulator of an activity rather than an inhibitor. Evidence presented herein that procaine and small molecules selected for their close chemical similarity to procaine lowered the hormone-stimulated corticosteroid formation by adrenal cells in vitro and in vivo by reducing the levels of the rate limiting enzyme HMG-CoA reductase mRNA, leading to reduced activity, and decreased cholesterol and corticosteroid biosynthesis and/or by regulating intracellular calcium concentration. These compounds do not affect basal corticosteroid formation, suggesting that only pathological states of high glucocorticoid formation would be affected. Such cortisol-modulating agents may be valuable for the treatment of high cortisol diseases such as, AIDS, multiple sclerosis, AD, depression, Cushing's hypertension either alone or in combination with disease-specific therapies.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for treatment of a mammal threatened or afflicted by an infectious pathogen, by administering to said mammal an effective amount of a compound of formula I: 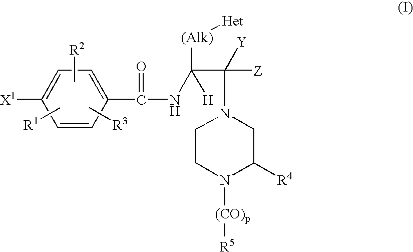
wherein:
a) R¹, R², R³, R⁴ and R⁵ are individually H. OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio or (C₁-C₆)alkanoyloxy; or R¹ and R² together are methylenedioxy;
b) X¹ is NO₂, CN, —N═O, (C₁-C₆)alkylC(O)NH—, isoxazolyl, or N(R⁶)(R⁷) wherein R⁶ and R⁷ are individually, H, (C₁-C₆)alkyl, (C₂-C₆)alkenyl, (C₃-C₆)cycloalkyl, (C₃-C₆)cycloalkyl((C₁-C₆)alkyl), wherein cycloalkyl optionally comprises 1-2, S, nonperoxide O or N(R⁸), wherein R⁸ is H, (C₁-C₆)alkyl, (C₃-C₆)cycloalkyl, (C₃-C₆)cycloalkyl(C₁-C₆)alkyl or benzyl; aryl, aryl(C₁-C₆)alkyl, aryl(C₂-C₆)alkenyl, heteroaryl, heteroaryl(C₁-C₆)alkyl, or R⁶ and R⁷ together with the N to which they are attached form a 5- or 6-membered heterocyclic or heteroaryl ring, optionally substituted with R¹ and optionally comprising 1-2, S. non-peroxide O or N(R⁵);
c) Alk is (C₁-C₆)alkyl;
d) Y and Z together are ═O, —O(CH₂)ₘO— or —(CH₂)ₘ— wherein m is 2-4, or Y is H and Z is OH or SH;
e) Het is heteroaryl or heterocycloalkyl, each optionally substituted by 1, 2 or 3 of R¹ or a combination thereof or is a bond connecting (Alk) to NH;
f) p is 0 or 1; or the pharmaceutically acceptable salt thereof.

2. The method of claim 1 wherein the amount is effective to inhibit entry of the pathogen or a subunit thereof into mammalian cells.

3. The method of claim 2 wherein the pathogen is a virus.

4. The method of claim 3 wherein the pathogen is a retrovirus.

5. The method of claim 4 wherein the pathogen is HIV.

6. The method of claim 1 wherein the pathogen is a bacterium.

7. The method of claim 1 wherein the cells are contacted in vitro.

8. The method of claim 2 wherein the cells are contacted in vivo.

9. The method of claim 8 wherein the compound of formula I is administered to a human.

10. The method of claim 9 wherein the human has been exposed to a virus.

11. The method of claim 9 wherein the human has been exposed to a retrovirus.

12. The method of claim 11 wherein the human is HIV-positive or is an AIDs patient.

13. The method of claim 1 wherein (Alk) is (C₁-C₄)alkyl.

14. The method of claim 1 wherein R⁴ and R⁵ are individually (C₁-C₆)alkyl, (C₃-C₆)cycloalkyl or (C₃-C₆)cycloalkyl(C₁-C₆)alkyl.

15. The method of claim 14 wherein R⁴ and R⁵ are (C₁-C₄)alkyl or (C₅-C₆)cycloalkyl.

16. The method of claim 1 wherein 1 or 2 of R¹, R² or R³ is H or (C₁-C₆)alkoxy.

17. The method of claim 16 wherein 1 or 2 of R¹, R² or R³ is (C₁-C₃)alkoxy.

18. The method of claim 1 wherein Y and Z together are ═O.

19. The method of claim 1 wherein p is 1.

20. The method of claim 1 wherein Het is 1H-indol-3-yl or imidazolin-3-yl.

21. The method of claim 1 wherein the compound of formula I is administered orally to a human.

22. The method of claim 1 wherein the compound of formula I is administered parenterally, as by injection, infusion, inhalation or insufflation, to a human.

23. The method of claim 1 wherein the compound of formula (I) is administered in combination with a pharmaceutically acceptable carrier.

24. The method of claim 23 wherein the carrier is a liquid.

25. The method of claim 23 wherein the carrier and the compound form a solution, a suspension or a gel.

26. The method of claim 23 wherein the carrier is a solid.

27. The method of claim 23 wherein the carrier comprises an effective amount of zinc sulfate heptahydrate.

28. The method of claim 1 wherein the compound of formula I is N-[2-((4-cyclopropylcarbonyl)-3-methylpiperazin-1-yl)-1-(1H-indol-3-yl-methyl)-2-(oxo)ethyl]-4-nitrobenzamide.

29. A method for treatment of a mammal threatened or afflicted by a neuropathological condition by administering to said mammal an effective neuroprotective amount of a compound of formula I: 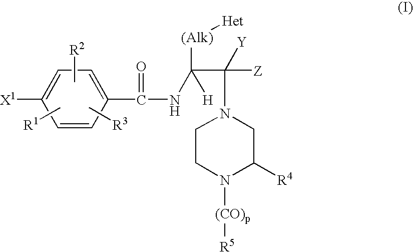
wherein:
a) R¹, R², R³, R⁴ and R⁵ are individually H, OH, halo, (C₁-C₆)alkyl, (C₁-C₆)alkoxy, (C₃-C₆)cycloalkyl, (C₃-C₆)cycloalkyl((C₁-C₆)alkyl), (C₂-C₆)alkenyl, (C₂-C₆)alkynyl, (C₁-C₆)alkanoyl, halo(C₁-C₆)alkyl, hydroxy(C₁-C₆)alkyl, (C₁-C₆)alkoxycarbonyl; (C₁-C₆)alkylthio or (C₁-C₆)alkanoyloxy; or R¹ and R² together are methylenedioxy;
b) X¹ is, NO₂, CN, —N═O, (C₁-C₆)alkyl(C(O)NH—, isoxazolyl, or N(R⁶)(R⁷) wherein R⁶ and R⁷ are individually, H, (C₁-C₆)alkyl, (C₂-C₆)alkenyl, (C₃-C₆)cycloalkyl, (C₃-C₆)cycloalkyl(C₁-C₆)alkyl), wherein cycloalkyl optionally comprises 1-2, S, nonperoxide O or N(R⁸), wherein R⁸is H, (C₁-C₆)alkyl, (C₃-C₆)cycloalkyl, (C₃-C₆)cycloalkyl(C₁-C₆)alkyl or benzyl; aryl, aryl(C₁-C₆)alkyl, aryl(C₂-C₆)alkenyl, heteroaryl, heteroaryl(C₁-C₆)alkyl, or R⁶ and R⁷ together with the N to which they are attached form a 5- or 6-membered heterocyclic or heteroaryl ring, optionally substituted with R¹ and optionally comprising 1-2, S, non-peroxide O or N(R⁵);
c) Alk is (C₁-C₆)alkyl;
d) Y and Z together are ═O, —O(CH₂)ₘO— or —(CH₂)ₘ— wherein m is 2-4, or Y is H and Z is OH or SH;
e) Het is heteroaryl or heterocycloalkyl, each optionally substituted by 1, 2 or 3 of R¹ or a combination thereof or is a bond connecting (Alk) to NH;
f) p is 0 or 1; and the pharmaceutically acceptable salts thereof.

30. The method of claim 29 wherein the amount is effective to treat at least one symptom of Alzheimer's disease or vascular dementia.

31. The method of claim 29 wherein the compound of formula (I) comprises 1-(4-cyclopropanecarbonyl-3-methyl-piperazine-1-carbonyl)-(1H-indol-3-yl-methyl)-(4-nitrobenzamido)-methane.

32. The method of claim 29 wherein the compound of formula (I) comprises (4-cyclopropanecarbonyl-3-methyl-piperazine-1-carbonyl)-2-(1H-indol-3-yl-methyl)-4-(4-nitrophenyl)-butane-1,4-dione.

33. A method of treating a neuropathological condition by administering to a subject in need thereof, an effective amount of acetic acid-4,5-diacetoxy-2-acetoxymethyl-6-[4-(2-diethylamino-ethylcarbamoyl)-2-methoxyphenoxy]-tetrahydro-pyran-3-yl ester.

34. A method of treating a neuropathological condition by administering to a subject in need thereof, an effective amount of acetic acid-5-acetoxy-3-(4-benzoyl-piperazin-1-yl-methyl)-4-hydroxy-4a,8-dimethyl-2-oxododecahydro-azuleno[6,5-b]furan-4-yl ester.

35. A method of treating a neuropathological condition by administering to a subject in need thereof, an effective amount of 3-(4-benzoyl-piperazin-1-yl-methyl)-6,6a-epoxy-6,9-dimethyl-3a,4,5,6,6a,7,9a,9b-octahydro-3H-azuleno[4,5-b]furan-2-one.

36. A method of treating a neuropathological condition by administering to a subject in need thereof an effective amount of procaine or a pharmaceutically acceptable salt thereof.

37. The method of claim 29 wherein (Alk) is (C₁-C₄)alkyl.

38. The method of claim 29 wherein R⁴ and R⁵ are individually (C₁-C₆)alkyl, (C₃-C₆)cycloalkyl or (C₃-C₆)cycloalkyl(C₁-C₆)alkyl.

39. The method of claim 29 wherein R⁴ and R⁵ are individually (C₁-C₄)alkyl or (C₃-C₆)cycloalkyl.

40. The method of claim 29 wherein 1 or 2 of R¹, R² or R³ is H or (C₁-C₆)alkoxy.

41. The method of claim 29 wherein Y and Z together are ═O.

42. The method of claim 29 wherein p is 1.

43. The method of of claim 29 wherein Het is 1H-indol-3-yl or imidazolin-3-yl.

44. The method of claim 29 wherein the compound of formula I is administered orally.

45. The method of claim 29 wherein the compound of formula (I) is administered by parenterally.

46. The method of claim 45 wherein the compound of formula I is administered by injection, infusion, inhalation or insufflation, to a mammal.

47. The method of claim 29 wherein the compound of formula (I) is administered in combination with a pharmaceutically acceptable carrier.

48. The method of claim 47 wherein the carrier is a liquid.

49. The method of claim 47 wherein the compound and the carrier form a solution, suspension or gel.

50. The method of claim 47 wherein the carrier is a solid.

51. The method of claim 29 wherein the compound of formula I is N-[2-((4-cyclopropylcarbonyl)-3-methylpiperazin-1-yl)-1-(1H-indol-3-yl-methyl)-2-(oxo)ethyl]-4-nitrobenzamide.

52. The method of claim 29 wherein the neuropathological condition is Alzheimer's disease.

53. The method of claim 29 wherein the amount is effective to inhibit Aβ peptide-induced neurotoxicity.

54. The method of claim 53 wherein the amount is effective to inhibit Aβ₁₋₄₀, Aβ₁₋₄₂ or Aβ₁₋₄₃ neurotoxicity.

55. The method of claim 29 wherein the amount is effective to inhibit glutamate-induced neurotoxicity.

56. The method of claim 29 wherein the neuropathological condition is due to hyper-stimulation of a glutamate pathway.

57. The method of claim 29 wherein the amount is effective to maintain ATP levels in neuronal cells.

58. The method of claim 29 wherein the compound of formula I is administered to a human.

59. The method of claim 58 wherein the human is in an early stage of AD.

60. The method of claim 58 wherein the human is an AD patient.

61. The method of claim 58 wherein the human is afflicted with vascular dementia.

62. A dosage form comprising a compound of formula (I) in combination with a pharmaceutically-acceptable carrier.
